(19) United States
(12) Patent Application Publication
(10) Pub. No.: US 2026/0232659 A1
(43) Pub. Date: Aug. 13, 2026

(54) CHECKPOINT KINASE 1 (CHK1) INHIBITOR PHARMACEUTICAL COMPOSITION

(71) Applicant: Boundless Bio, Inc., San Diego, CA (US)

(72) Inventors: Eric F. CONNOR, San Jose, CA (US); Anthony B. PINKERTON, Rancho Santa Fe, CA (US); Stephen Todd MEYER, San Diego, CA (US)

(21) Appl. No.: 19/471,099

(22) PCT Filed: Apr. 2, 2024
(86) PCT No.: PCT/US2024/022615

§ 371 Date: Sep. 30, 2025

Related U.S. Application Data
(60) Provisional application No. 63/493,847, filed on Apr. 3, 2023.

(51) Int. Cl. A61K 31/497; A61K 31/506; A61K 31/517; A61K 31/519; A61K 31/5377; A61P 35/00

(52) U.S. Cl. CPC A61K 31/497; A61K 31/506; A61K 31/517; A61K 31/519; A61K 31/5377; A61P 35/00

(57) ABSTRACT

Provided herein is a pharmaceutical composition Compound (1) comprising a CHK1 inhibitor.

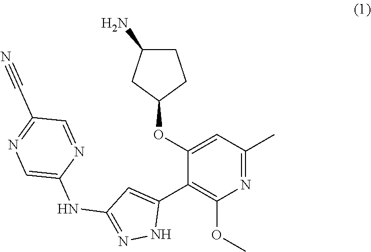 [SMILES: COc1nc(C)cc(O[C@@H]2CC[C@H](N)C2)c1-c1cc(Nc2cnc(C#N)cn2)n[nH]1]

### CROSS-REFERENCE

This application claims the benefit of U.S. Provisional Application Ser. No. 63/493,847 filed Apr. 3, 2023 which is hereby incorporated by reference in its entirety.

### BACKGROUND OF THE INVENTION

Cancer remains the second leading cause of death in the United States (US), accounting for approximately 1,900,000 new diagnoses and 610,000 deaths on an annual basis. Globally, cancer is the second leading cause of death, and was responsible for nearly 10 million deaths in 2020; nearly 1 in 6 deaths was due to cancer. The number of new cases is expected to rise by 70% over the next 2 decades.

In solid malignancies, metastatic spread, and systemic disease accounts for approximately 90% of cancer-related deaths. Despite progress made over the past few decades, there remains a need for the development of targeted interventions for advanced or metastatic solid tumors.

Patients with cancers that harbor oncogene amplifications, with the exception of HER2 (or ERBB2), have no targeted therapies approved as standard of care. Generally, these patients suffer worse survival rates than patients with other forms of oncogene alteration or with no known oncogene alteration.

There are currently no therapies approved to treat patients with these other oncogene-amplified tumors, and especially no oral therapy.

### BRIEF SUMMARY OF THE INVENTION

Disclosed herein is a pharmaceutical composition comprising:
(a) about 5% to about 20% (w/w) 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

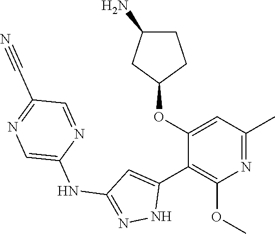 [SMILES: COc1nc(C)cc(O[C@@H]2CC[C@H](N)C2)c1-c1cc(Nc2cnc(C#N)cn2)n[nH]1]

(Compound 1) or a pharmaceutically acceptable salt thereof;
(b) about 70% to about 90% (w/w) of a diluent;
(c) about 1% to about 10% (w/w) of a disintegrant;
(d) about 0.1% to about 2% of a glidant; and
(e) about 0.1% to about 2% of a lubricant.

In some embodiments of a pharmaceutical composition, the amount of the diluent in the pharmaceutical composition is between about 80% and about 90% (w/w).

In some embodiments of a pharmaceutical composition, the amount of the diluent in the pharmaceutical composition is about 85% (w/w).

In some embodiments of a pharmaceutical composition, the diluent is microcrystalline cellulose.

In some embodiments of a pharmaceutical composition, the amount of the disintegrant in the pharmaceutical composition is between about 2% to about 4% (w/w).

In some embodiments of a pharmaceutical composition, the amount of the disintegrant in the pharmaceutical composition is about 3% (w/w).

In some embodiments of a pharmaceutical composition, the disintegrant is croscarmellose sodium.

In some embodiments of a pharmaceutical composition, the amount of the glidant in the pharmaceutical composition is between about 0.5% to about 1.5% (w/w).

In some embodiments of a pharmaceutical composition, the amount of the glidant in the pharmaceutical composition is about 1% (w/w).

In some embodiments of a pharmaceutical composition, the glidant is colloidal silicon dioxide.

In some embodiments of a pharmaceutical composition, the amount of the lubricant in the pharmaceutical composition is between about 0.5% to about 1.5% (w/w).

In some embodiments of a pharmaceutical composition, the amount of the lubricant in the pharmaceutical composition is about 1% (w/w).

In some embodiments of a pharmaceutical composition, the lubricant is magnesium stearate.

In some embodiments of a pharmaceutical composition, the composition further comprises a coating.

In some embodiments of a pharmaceutical composition, the coating comprises a film former, a plasticizer, an opacifying agent, a colorant, or any combination thereof.

In some embodiments of a pharmaceutical composition, the pharmaceutical composition is stable at 25° C./60% RH.

In some embodiments of a pharmaceutical composition, the pharmaceutical composition is stable at 40° C./75% RH.

In some embodiments of a pharmaceutical composition, the pharmaceutical composition is stable at 2-8° C.

In some embodiments of a pharmaceutical composition, the pharmaceutical composition is comprised in a tablet.

In some embodiments of a pharmaceutical composition, the pharmaceutical composition is comprised in a 5 mg tablet or a 20 mg tablet.

Also disclosed herein is a method of treating cancer in a subject in need thereof comprising administering to the subject a pharmaceutical composition disclosed herein.

In some embodiments, the cancer comprises a solid tumor.

In some embodiments, the cancer comprises a locally advanced or metastatic non-resectable solid tumor.

In some embodiments, the cancer comprises a tumor or tumor cells harboring an oncogene amplification.

In some embodiments, the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

In some embodiments, the oncogene amplification resides on ecDNA.

In some embodiments, the oncogene amplification resides on one or more chromosomal loci.

In some embodiments, the oncogene amplification is an ecDNA-derived amplification.

In some embodiments, the pharmaceutical composition is administered every other day.

In some embodiments, the cancer is an ovarian cancer.

In some embodiments, the ovarian cancer is a platinum resistant high-grade serous ovarian cancer, a primary peritoneal cancer, or a fallopian tube cancer.

In some embodiments, the cancer is a uterine cancer.

In some embodiments, the uterine cancer is a high-grade endometrial carcinoma, a uterine serous carcinoma or a uterine carcinosarcoma.

In some embodiments, the cancer is colorectal cancer, esophageal cancer, gastric cancer, gastroesophageal junction (GEJ) cancer, head and neck squamous cell carcinoma, liposarcoma, non-small cell lung cancer, or subtype squamous cell carcinoma.

In some embodiments, the cancer is glioblastoma or a neuroblastoma.

In some embodiments, the cancer is breast cancer, cholangiocarcinoma, esophageal cancer, neck squamous cell carcinoma, non-small cell lung cancer, stomach cancer, or subtype squamous cell carcinoma.

In some embodiments, the cancer is esophageal cancer, non-small cell lung cancer, a sarcoma, or stomach cancer.

In some embodiments, the treatment further comprises administering an additional therapeutic agent.

In some embodiments, the oncogene amplification comprises CDK4, CDK6, EGFR, FGFR1, FGFR2, or FGFR3.

In some embodiments, the treatment further comprises administering a CDK4/6 inhibitor, an EGFR inhibitor, or a FGFR inhibitor. In some embodiments, the EGFR inhibitor is erlotinib. In some embodiments, the FGFR inhibitor is pemigatinib. In some embodiments, the FGFR inhibitor is futibatinib. In some embodiments, the CDK4/6 inhibitor is abemaciclib.

In some embodiments, the pharmaceutical composition of Compound 1 and the additional therapeutic agent are each formulated for oral administration.

### INCORPORATION BY REFERENCE

All publications, patents, and patent applications mentioned in this specification are herein incorporated by reference for the specific purposes identified herein.

### DETAILED DESCRIPTION OF THE INVENTION

Focal high-copy number oncogene amplifications are frequently observed to occur on extrachromosomal DNA (ecDNA). ecDNA are found in tumor cells and are derived from extrachromosomal fragments of genomic DNA that often encode full length genes and regulatory regions such as promoters. ecDNA may be found as physically distinct from chromosomes and are engendered with unique properties, including an open chromatin architecture associated with hyper-transcription and a predilection for structural variation. In addition, because ecDNA lack centromeres, extrachromosomally located ecDNA are inherited during cellular division via acentric, non-Mendelian segregation, which enables high copy number gene heterogeneity across the tumor cell population. Due to these properties, ecDNA are a common cellular mechanism for oncogene amplification (e.g., EGFR), and they facilitate hyper-transcription and overexpression of oncoproteins, which drive tumor growth and survival. Moreover, these features afford oncogene amplified ecDNA-enabled tumor cells with unparalleled genomic plasticity, facilitating both oncogenesis and circumvention of therapeutic pressure through rapid genomic evolution. Cancer cells that harbor oncogene amplifications on ecDNA bear high levels of intrinsic DNA replication stress (RS). The checkpoint kinase 1 (CHK1) serves an essential role in managing RS, making CHK1 a potential therapeutic target for cancers that have intrinsic elevated RS, including those with ecDNA-enabled oncogenic amplifications. Consistent with this hypothesis, ecDNA-enabled oncogene-amplified tumor cells display enhanced sensitivity to CHK1 inhibition when compared to ecDNA negative non-amplified cells. Applying targeted therapy (e.g., EGFR inhibitor) pressure to the protein products of oncogenes (e.g., EGFR) amplified on ecDNA induces cancer cells to evade such pressure, and these resistance mechanisms further increase RS and reliance upon CHK1. Accordingly, combining targeted therapy pressure (e.g., EGFR inhibitor) with CHK1 pressure (i.e., CHK1 inhibitor) in ecDNA-enabled tumor cells provides a synergistic therapeutic effect. There is a need therefore, as recognized and addressed herein, to provide CHK1 inhibitors with desired clinical and therapeutic properties, including inhibitors and formulations that are orally available therapies.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment.

Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms “treat,” “treated,” “treatment,” or “treating” as used herein refers to therapeutic treatment, wherein the object is to prevent or slow (lessen) an undesired physiological condition, disorder, or disease, or to obtain beneficial or desired clinical results. For the purposes described herein, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. The terms “treat,” “treated,” “treatment,” or “treating” as well as words stemming therefrom, as used herein, do not necessarily imply 100% or complete treatment. Rather, there are varying degrees of treatment of which one of ordinary skill in the art recognizes as having a potential benefit or therapeutic effect. In this respect, the disclosed methods can provide any amount of any level of treatment of the disorder in a mammal. For example, a disorder, including symptoms or conditions thereof, may be reduced by, for example, about 100%, about 90%, about 80%, about 70%, about 60%, about 50%, about 40%, about 30%, about 20%, or about 10%.

The terms “effective amount” or “therapeutically effective amount,” as used herein, refer to a sufficient amount of a compound disclosed herein being administered which will relieve to some extent one or more of the symptoms of the disease or condition being treated, e.g., cancer or an inflammatory disease. In some embodiments, the result is a reduction and/or alleviation of the signs, symptoms, or causes of a disease, or any other desired alteration of a biological system. For example, an “effective amount” for therapeutic uses is the amount of the composition comprising a compound disclosed herein required to provide a clinically significant decrease in disease symptoms. In some embodiments, an appropriate “effective” amount in any individual case is determined using techniques, such as a dose escalation study.

The term “biological sample,” as used herein, generally refers to a sample derived from or obtained from a subject, such as a mammal (e.g., a human). Biological samples are contemplated to include but are not limited to, hair, fingernails, skin, sweat, tears, ocular fluids, nasal swab or nasopharyngeal wash, sputum, throat swab, saliva, mucus, blood, serum, plasma, placental fluid, amniotic fluid, cord blood, emphatic fluids, cavity fluids, earwax, oil, glandular secretions, bile, lymph, pus, microbiota, meconium, breast milk, bone marrow, bone, CNS tissue, cerebrospinal fluid, adipose tissue, synovial fluid, stool, gastric fluid, urine, semen, vaginal secretions, stomach, small intestine, large intestine, rectum, pancreas, liver, kidney, bladder, lung, and other tissues and fluids derived from or obtained from a subject.

The term “tumor” or “tumor cells” as used herein, generally refers to cells that grow and divide more than they should or do not die when they should. In some cases, tumor cells are present in a solid mass, such as a solid tumor, or in some cases, tumor cells are found in a non-solid form, such as in blood cancers. Tumor or tumor cells also can include metastasis or metastasizing cells, where cancer cells break away from the original (primary) tumor and may form a new tumor in other organs or tissues of the body.

The term “ecDNA signature” as used herein, generally refers to one or more characteristics common to tumors or tumor cells that are ecDNA+. In some cases, the ecDNA signature is selected from the group consisting of a gene amplification; a p53 loss of function mutation; absence of microsatellite instability (MSI-H); a low level of PD-L1 expression; a low level of tumor inflammation signature (TIS); a low level of tumor mutational burden (TMB); an increased frequency of allele substitutions, insertions, or deletions (indels); and any combination thereof. In some cases, the ecDNA signature can include an increase in copy number (gene amplification) in conjunction with particular structural variations. In some cases, the ecDNA signature can include a focal amplification. In some cases, ecDNA signature includes a detection or identification of ecDNA using an imaging technology. In some cases, ecDNA signature does not include any imaging or direct detection of ecDNA.

### Compounds

Described herein are method of treating cancer in a subject in need thereof comprising administering to the subject a CHK1 inhibitor.

### Compound 1

In some embodiments, the CHK1 inhibitor is Compound 1, or a pharmaceutically acceptable salt thereof. Compound 1 is 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

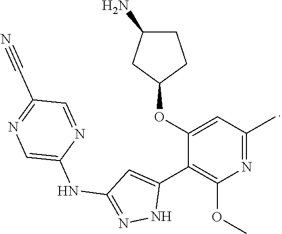 [SMILES: COc1nc(C)cc(O[C@@H]2CC[C@H](N)C2)c1-c1cc(Nc2cnc(C#N)cn2)n[nH]1]

In some embodiments, Compound 1 is a free base.

### Pharmaceutically Acceptable Salts

In some embodiments, Compound 1 described herein exists as its pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

Examples of pharmaceutically acceptable salts include those salts prepared by reaction of the compounds described herein with a mineral, organic acid, or inorganic base, such salts including acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undecanoate, and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Pharmaceutical Composition

Disclosed herein is a pharmaceutical composition comprising 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

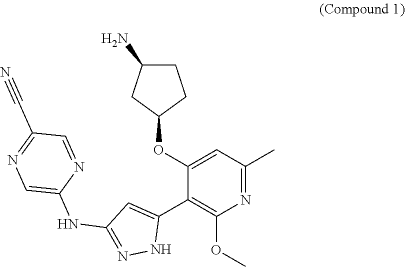 [SMILES: COc1nc(C)cc(O[C@@H]2CC[C@H](N)C2)c1-c1cc(Nc2cnc(C#N)cn2)n[nH]1]

or a pharmaceutically acceptable salt thereof. Disclosed herein is a pharmaceutical composition comprising 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

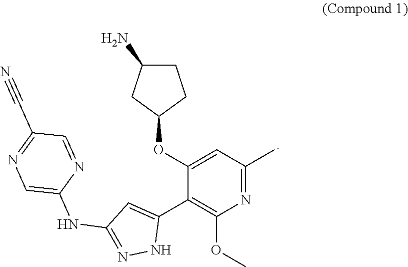 [SMILES: COc1nc(C)cc(O[C@@H]2CC[C@H](N)C2)c1-c1cc(Nc2cnc(C#N)cn2)n[nH]1]

In some embodiments, the pharmaceutical composition is for oral administration. In some embodiments, the pharmaceutical composition is in a suitable form that can be stored in bottles and used at home. In some embodiments, the pharmaceutical composition is in a form of a tablet. In some embodiments, the pharmaceutical composition is stable (non-hydroscopic, not discolored). In some embodiments, the pharmaceutical composition comprises stable and non-toxic pharmaceutically acceptable excipients. In some embodiments, the pharmaceutical composition is formulated for immediate release. In some embodiments, the pharmaceutical composition is formulated as an immediate release (IR) film coated tablet. In some embodiments, the process of making the pharmaceutical composition does not introduce any unwanted impurities.

Disclosed herein is a pharmaceutical composition comprising:
(a) about 5% to about 20% (w/w) 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

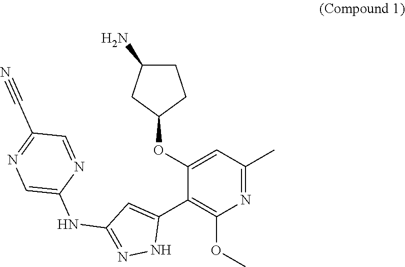 [SMILES: COc1nc(C)cc(O[C@@H]2CC[C@H](N)C2)c1-c1cc(Nc2cnc(C#N)cn2)n[nH]1]

or a pharmaceutically acceptable salt thereof;
(b) about 70% to about 90% (w/w) of a diluent;
(c) about 1% to about 10% (w/w) of a disintegrant;
(d) about 0.1% to about 2% of a glidant; and
(e) about 0.1% to about 2% of a lubricant.

Disclosed herein is a pharmaceutical composition comprising:
(a) about 5% to about 20% (w/w) 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

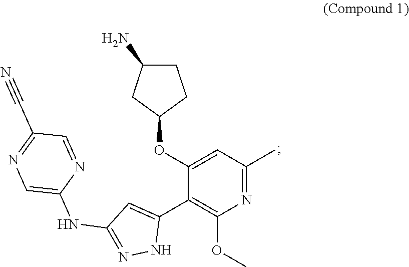 [SMILES: COc1nc(C)cc(O[C@@H]2CC[C@H](N)C2)c1-c1cc(Nc2cnc(C#N)cn2)n[nH]1]

(b) about 70% to about 90% (w/w) of a diluent;
(c) about 1% to about 10% (w/w) of a disintegrant;
(d) about 0.1% to about 2% of a glidant; and
(e) about 0.1% to about 2% of a lubricant.

In some embodiments of the pharmaceutical composition, the amount of the diluent in the pharmaceutical composition is between about 80% and about 90% (w/w).

In some embodiments of the pharmaceutical composition, the amount of the diluent in the pharmaceutical composition is about 80% (w/w). In some embodiments of the pharmaceutical composition, the amount of the diluent in the pharmaceutical composition is about 81% (w/w). In some embodiments of the pharmaceutical composition, the amount of the diluent in the pharmaceutical composition is about 82% (w/w). In some embodiments of the pharmaceutical composition, the amount of the diluent in the pharmaceutical composition is about 83% (w/w). In some embodiments of the pharmaceutical composition, the amount of the diluent in the pharmaceutical composition is about 84% (w/w). In some embodiments of the pharmaceutical composition, the amount of the diluent in the pharmaceutical composition is about 85% (w/w). In some embodiments of the pharmaceutical composition, the amount of the diluent in the pharmaceutical composition is about 86% (w/w). In some embodiments of the pharmaceutical composition, the amount of the diluent in the pharmaceutical composition is about 87% (w/w). In some embodiments of the pharmaceutical composition, the amount of the diluent in the pharmaceutical composition is about 88% (w/w). In some embodiments of the pharmaceutical composition, the amount of the diluent in the pharmaceutical composition is about 89% (w/w). In some embodiments of the pharmaceutical composition, the amount of the diluent in the pharmaceutical composition is about 90% (w/w).

In some embodiments of the pharmaceutical composition, the diluent is microcrystalline cellulose, lactose, mannitol, or dicalcium phosphate.

In some embodiments of the pharmaceutical composition, the diluent is microcrystalline cellulose.

In some embodiments of the pharmaceutical composition, the amount of the disintegrant in the pharmaceutical composition is between about 2% to about 4% (w/w).

In some embodiments of the pharmaceutical composition, the amount of the disintegrant in the pharmaceutical composition is about 2% (w/w). In some embodiments of the pharmaceutical composition, the amount of the disintegrant in the pharmaceutical composition is about 2.5% (w/w). In some embodiments of the pharmaceutical composition, the amount of the disintegrant in the pharmaceutical composition is about 3% (w/w). In some embodiments of the pharmaceutical composition, the amount of the disintegrant in the pharmaceutical composition is about 3.5% (w/w). In some embodiments of the pharmaceutical composition, the amount of the disintegrant in the pharmaceutical composition is about 4% (w/w).

In some embodiments of the pharmaceutical composition, the disintegrant is croscarmellose sodium, sodium starch glycolate, starch, or crosslinked-polyvinylpyroidine (cross-PVP).

In some embodiments of the pharmaceutical composition, the disintegrant is croscarmellose sodium.

In some embodiments of the pharmaceutical composition, the amount of the glidant in the pharmaceutical composition is between about 0.5% to about 1.5% (w/w).

In some embodiments of the pharmaceutical composition, the amount of the glidant in the pharmaceutical composition is about 0.5% (w/w). In some embodiments of the pharmaceutical composition, the amount of the glidant in the pharmaceutical composition is about 0.6% (w/w). In some embodiments of the pharmaceutical composition, the amount of the glidant in the pharmaceutical composition is about 0.7% (w/w). In some embodiments of the pharmaceutical composition, the amount of the glidant in the pharmaceutical composition is about 0.8% (w/w). In some embodiments of the pharmaceutical composition, the amount of the glidant in the pharmaceutical composition is about 0.9% (w/w). In some embodiments of the pharmaceutical composition, the amount of the glidant in the pharmaceutical composition is about 1% (w/w). In some embodiments of the pharmaceutical composition, the amount of the glidant in the pharmaceutical composition is about 1.1% (w/w). In some embodiments of the pharmaceutical composition, the amount of the glidant in the pharmaceutical composition is about 1.2% (w/w). In some embodiments of the pharmaceutical composition, the amount of the glidant in the pharmaceutical composition is about 1.3% (w/w). In some embodiments of the pharmaceutical composition, the amount of the glidant in the pharmaceutical composition is about 1.4% (w/w). In some embodiments of the pharmaceutical composition, the amount of the glidant in the pharmaceutical composition is about 1.5% (w/w).

In some embodiments of the pharmaceutical composition, the glidant is colloidal silicon dioxide, silicon dioxide, talc, magnesium stearate, magnesium carbonate, or fumed silica.

In some embodiments of the pharmaceutical composition, the glidant is colloidal silicon dioxide.

In some embodiments of the pharmaceutical composition, the amount of the lubricant in the pharmaceutical composition is between about 0.5% to about 1.5% (w/w).

In some embodiments of the pharmaceutical composition, the amount of the lubricant in the pharmaceutical composition is about 0.5% (w/w). In some embodiments of the pharmaceutical composition, the amount of the lubricant in the pharmaceutical composition is about 0.6% (w/w). In some embodiments of the pharmaceutical composition, the amount of the lubricant in the pharmaceutical composition is about 0.7% (w/w). In some embodiments of the pharmaceutical composition, the amount of the lubricant in the pharmaceutical composition is about 0.8% (w/w). In some embodiments of the pharmaceutical composition, the amount of the lubricant in the pharmaceutical composition is about 0.9% (w/w). In some embodiments of the pharmaceutical composition, the amount of the lubricant in the pharmaceutical composition is about 1% (w/w). In some embodiments of the pharmaceutical composition, the amount of the lubricant in the pharmaceutical composition is about 1.1% (w/w). In some embodiments of the pharmaceutical composition, the amount of the lubricant in the pharmaceutical composition is about 1.2% (w/w). In some embodiments of the pharmaceutical composition, the amount of the lubricant in the pharmaceutical composition is about 1.3% (w/w). In some embodiments of the pharmaceutical composition, the amount of the lubricant in the pharmaceutical composition is about 1.4% (w/w). In some embodiments of the pharmaceutical composition, the amount of the lubricant in the pharmaceutical composition is about 1.5% (w/w).

In some embodiments of the pharmaceutical composition, the lubricant is magnesium stearate, calcium stearate, stearic acid, or sodium stearyl fumarate.

In some embodiments of the pharmaceutical composition, the lubricant is magnesium stearate.

Disclosed herein is a pharmaceutical composition comprising:
(a) about 5% to about 20% (w/w) 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

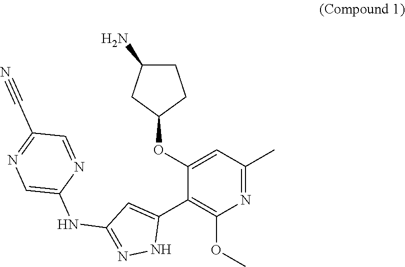 [SMILES: COc1nc(C)cc(O[C@@H]2CC[C@H](N)C2)c1-c1cc(Nc2cnc(C#N)cn2)n[nH]1]

or a pharmaceutically acceptable salt thereof;
(b) about 70% to about 90% (w/w) of microcrystalline cellulose;
(c) about 1% to about 10% (w/w) of croscarmellose sodium;
(d) about 0.1% to about 2% of colloidal silicon dioxide; and
(e) about 0.1% to about 2% of magnesium stearate.

Disclosed herein is a pharmaceutical composition comprising:
(a) about 5% to about 20% (w/w) 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

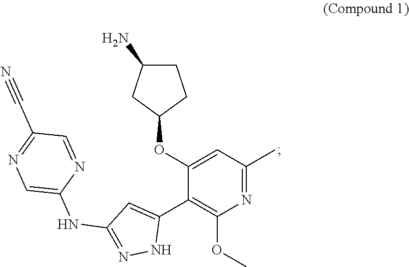 [SMILES: COc1nc(C)cc(O[C@@H]2CC[C@H](N)C2)c1-c1cc(Nc2cnc(C#N)cn2)n[nH]1]

(b) about 70% to about 90% (w/w) of microcrystalline cellulose;
(c) about 1% to about 10% (w/w) of croscarmellose sodium;
(d) about 0.1% to about 2% of colloidal silicon dioxide; and
(e) about 0.1% to about 2% of magnesium stearate.

Disclosed herein is a pharmaceutical composition comprising:
(a) about 5 mg of 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

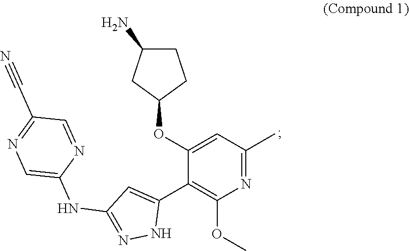 [SMILES: COc1nc(C)cc(O[C@@H]2CC[C@H](N)C2)c1-c1cc(Nc2cnc(C#N)cn2)n[nH]1]

(b) about 42.5 mg of microcrystalline cellulose;
(c) about 1.5 mg of croscarmellose sodium;
(d) about 0.5 mg of colloidal silicon dioxide; and
(e) about 0.5 mg of magnesium stearate.

Disclosed herein is a pharmaceutical composition comprising:
(a) about 20 mg of 5-((5-(4-(((1R,3S)-3-aminocyclopentyl)oxy)-2-methoxy-6-methylpyridin-3-yl)-1H-pyrazol-3-yl)amino)pyrazine-2-carbonitrile:

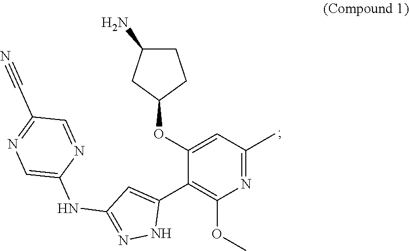 [SMILES: COc1nc(C)cc(O[C@@H]2CC[C@H](N)C2)c1-c1cc(Nc2cnc(C#N)cn2)n[nH]1]

(b) about 107 mg of microcrystalline cellulose;
(c) about 6.0 mg of croscarmellose sodium;
(d) about 2.0 mg of colloidal silicon dioxide; and
(e) about 2.0 mg of magnesium stearate.

In some embodiments of the pharmaceutical composition, the pharmaceutical composition further comprising a coating.

In some embodiments of the pharmaceutical composition, the coating comprises a film former, a plasticizer, an opacifying agent, a colorant, or any combination thereof.

In some embodiments of the pharmaceutical composition, the pharmaceutical composition is stable at 25° C./60% RH.

In some embodiments of the pharmaceutical composition, the pharmaceutical composition is stable at 40° C./75% RH.

In some embodiments of the pharmaceutical composition, the pharmaceutical composition is stable at 2-8° C.

### Methods

Disclosed herein is a method of treating cancer in a subject in need thereof comprising administering to the subject a pharmaceutical composition comprising Compound 1, or a pharmaceutically acceptable salt thereof. In some embodiments, the CHK1 inhibitor is Compound 1, or a pharmaceutically acceptable salt thereof.

In some embodiments of a method disclosed herein, the subject experiences a therapeutic response.

In some embodiments of a method disclosed herein, the therapeutic response comprises a reduction in the level of oncogene amplification in the tumor or tumor cells after treatment as compared to the level of oncogene amplification in the tumor or tumor cells prior to treatment.

In some embodiments of a method disclosed herein, the therapeutic response comprises reduction in of one or more of tumor growth, tumor size, number of tumor cells, or tumor metastasis as compared to prior to treatment.

In some embodiments of a method disclosed herein, the therapeutic response comprises a therapeutic benefit. In some embodiments of a method disclosed herein, the therapeutic benefit is stable disease (SD). In some embodiments of a method disclosed herein, the therapeutic benefit is partial response (PR). In some embodiments of a method disclosed herein, the therapeutic benefit is complete response (CR). In some embodiments of a method disclosed herein, a complete response is determined by RECISTv1.1 (or RANO for GBM). In some embodiments of a method disclosed herein, the therapeutic benefit is duration of response (DOR). In some embodiments of a method disclosed herein, the therapeutic benefit is progression-free survival (PFS). In some embodiments of a method disclosed herein, the therapeutic benefit is overall survival (OS).

In some embodiments of a method disclosed herein, the cancer comprises a solid tumor.

In some embodiments of a method disclosed herein, the cancer comprises a locally advanced or metastatic non-resectable solid tumor.

In some embodiments of a method disclosed herein, the cancer comprises a tumor or tumor cells harboring an oncogene amplification.

In some embodiments of a method disclosed herein, the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

In some embodiments of a method disclosed herein, the oncogene amplification comprises an amplification of FGFR1, FGFR2, FGFR3 or a combination thereof. In some embodiments of a method disclosed herein, the oncogene amplification comprises an amplification of FGFR1, FGFR2, FGFR3 or a combination thereof and wherein the treatment further comprises administering an FGFR inhibitor.

In some embodiments of a method disclosed herein, the oncogene amplification comprises CDK4, CDK6, EGFR, FGFR1, FGFR2, or FGFR3.

In some embodiments of a method disclosed herein, the oncogene amplification comprises an amplification of EGFR. In some embodiments of a method disclosed herein, the oncogene amplification comprises an amplification of EGFR and wherein the treatment further comprises administering an EGFR inhibitor.

In some embodiments of a method disclosed herein, the oncogene amplification comprises an amplification of CDK4, CDK6, or a combination thereof. In some embodiments of a method disclosed herein, the oncogene amplification comprises an amplification of CDK4, CDK6, or a combination thereof and wherein the treatment further comprises administering a CDK4/6 inhibitor.

In some embodiments of a method disclosed herein, the oncogene amplification resides on ecDNA.

In some embodiments of a method disclosed herein, the oncogene amplification resides on one or more chromosomal loci.

In some embodiments of a method disclosed herein, the oncogene amplification is an ecDNA-derived amplification.

In some embodiments of a method disclosed herein, the oncogene amplification has a copy number of at least 6, at least 8, at least 10, at least 15, at least 20 or more than 20 copies of the oncogene or portion thereof.

In some embodiments of a method disclosed herein, the cancer includes malignant tumors whose size can be decreased, whose growth or spread can be slowed or halted, or whose symptom is in remission or alleviated, reduced, and/or completely cured by deleting or suppressing and/or inhibiting functions of CHK1. Malignant tumors of interest are, but not limited to, head and neck cancer, gastrointestinal cancer (esophageal cancer, gastric cancer, duodenal cancer, liver cancer, biliary tract cancer (gallbladder, bile duct cancer, etc.), pancreatic cancer, colorectal cancer (colon cancer, rectal cancer, etc.), etc.), lung cancer (non-small cell lung cancer, small cell lung cancer, squamous cell lung carcinoma, lung adenocarcinoma, mesothelioma, etc.), breast cancer, genital cancer (ovarian cancer, uterine cancer, cervical cancer, endometrial cancer, etc.), urinary cancer (kidney cancer, bladder cancer, prostate cancer, testicular tumor, etc.), hematopoietic tumors (leukemia, malignant lymphoma, multiple myeloma, etc.), bone and soft tissue tumors (e.g., soft tissue sarcomas, liposarcoma, and osteosarcomas), skin cancer, brain tumor (e.g., glioblastoma) and the like.

In some embodiments of a method disclosed herein, the term cancer is used in accordance with its plain ordinary meaning in light of the present disclosure and refers to all types of cancer, neoplasm or malignant tumors found in mammals, including leukemias, lymphomas, melanomas, neuroendocrine tumors, carcinomas, and sarcomas. Exemplary cancers that may be treated with a compound disclosed herein, or a pharmaceutically acceptable salt, solvate, tautomer, or stereoisomer thereof, pharmaceutical compositions include acute myeloid leukemia, adrenal cortical cancer, adrenal gland cancer, bladder cancer, bone cancer, brain cancer, breast cancer (e.g., ductal carcinoma, lobular carcinoma, primary, metastatic), breast cancer, cancer of the endocrine system, cancer of the hepatic stellate cells, cancer of the pancreatic stellate cells, cervical cancer, colon cancer, colorectal cancer, ductal carcinoma, endometrial cancer, esophageal cancer, gastric cancer, genitourinary tract cancer, glioblastoma, glioma, head and neck cancer, hepatocellular carcinoma, Hodgkin's Disease, kidney cancer, leukemia (e.g., lymphoblastic leukemia, chronic lymphocytic leukemia, hairy cell leukemia), liver cancer (e.g., hepatocellular carcinoma), lobular carcinoma, lung cancer (e.g., non-small cell lung carcinoma, squamous cell lung carcinoma, adenocarcinoma, large cell lung carcinoma, small cell lung carcinoma, carcinoid, sarcoma), liposarcoma, lymph node cancer, lymphoma (e.g., Mantel cell lymphoma, follicular lymphoma, diffuse large B-cell lymphoma, marginal zona lymphoma, Burkitt's lymphoma, Non-Hodgkin's Lymphoma), malignant carcinoid, malignant hypercalcemia, malignant pancreatic insulinoma, medullary thyroid cancer, medulloblastoma, melanoma, mesothelioma, multiple myeloma muscle cancer, neoplasms of the endocrine or exocrine pancreas, neuroblastoma, ovarian cancer, Paget's Disease of the Nipple, pancreatic cancer, papillary thyroid cancer, Phyllodes Tumors, premalignant skin lesions, primary thrombocytosis, prostate cancer (e.g. castration-resistant prostate cancer), renal carcinoma, rhabdomyosarcoma, salivary gland cancer, sarcoma, soft tissue sarcoma, squamous cell carcinoma (e.g., head, neck, or esophagus), stomach cancer, testicular cancer, thyroid cancer, urinary bladder cancer, or uterine cancer. In embodiments, the cancer is selected from bladder cancer, breast cancer, colon cancer, esophageal cancer, esophageal cancer, glioblastoma, liver cancer, lung cancer, ovarian cancer, pancreatic cancer, prostate cancer, salivary gland cancer, soft tissue sarcoma, squamous cell lung carcinoma, stomach cancer, and uterine cancer.

In some embodiments of a method disclosed herein, the cancer is an ovarian cancer. In some embodiments of a method disclosed herein, the ovarian cancer is a platinum resistant high-grade serous ovarian cancer, a primary peritoneal cancer, or a fallopian tube cancer.

In some embodiments of a method disclosed herein, the cancer is a uterine cancer. In some embodiments of a method disclosed herein, the uterine cancer is a high-grade endometrial carcinoma, a uterine serous carcinoma or a uterine carcinosarcoma.

In some embodiments of a method disclosed herein, the cancer is glioblastoma or a neuroblastoma.

In some embodiments of a method disclosed herein, the cancer is colorectal cancer, esophageal cancer, gastric cancer, gastroesophageal junction (GEJ) cancer, head and neck squamous cell carcinoma, liposarcoma, non-small cell lung cancer, or subtype squamous cell carcinoma.

In some embodiments of a method disclosed herein, the cancer is breast cancer or head, esophageal cancer, neck squamous cell carcinoma, non-small cell lung cancer, stomach cancer, or subtype squamous cell carcinoma.

In some embodiments of a method disclosed herein, the cancer is esophageal cancer, non-small cell lung cancer, a sarcoma, or stomach cancer. In some embodiments of a method disclosed herein, the cancer is esophageal cancer. In some embodiments of a method disclosed herein, the cancer is non-small cell lung cancer. In some embodiments of a method disclosed herein, the cancer is a sarcoma. In some embodiments of a method disclosed herein, the cancer is stomach cancer.

In some embodiments of a method disclosed herein, the subject has undergone one or more prior therapies.

In some embodiments of a method disclosed herein, the subject was non-responsive to the one or more prior therapies.

In some embodiments of a method disclosed herein, the subject developed resistance to the one or more prior therapies.

In some embodiments of a method disclosed herein, the one or more prior therapies is chemotherapies.

In some embodiments of a method disclosed herein, the one or more prior therapies is a PD1 antibody.

In some embodiments of a method disclosed herein, the one or more prior therapies is a PD-L1 antibody.

In some embodiments of a method disclosed herein, the one or more prior therapies is a CTLA4 checkpoint inhibitor.

In some embodiments of a method disclosed herein, the one or more prior therapies is VEGF targeting therapies (e.g., bevacizumab for ovarian cancer).

Disclosed herein is a method of treating cancer in a subject in need thereof comprising administering to the subject a pharmaceutical composition comprising Compound 1, or a pharmaceutically acceptable salt thereof, wherein the method further comprises obtaining a diagnostic indicator of oncogene amplification in a biological sample from the subject.

In some embodiments of a method disclosed herein, the diagnostic indicator is obtained prior to a first administration of Compound 1, or a pharmaceutically acceptable salt thereof.

In some embodiments of a method disclosed herein, the diagnostic indicator is obtained subsequent to a first administration of Compound 1, or a pharmaceutically acceptable salt thereof.

In some embodiments of a method disclosed herein, the diagnostic indicator is obtained subsequent to multiple administrations of Compound 1, or a pharmaceutically acceptable salt thereof.

In some embodiments of a method disclosed herein, the diagnostic indicator results from a next generation sequencing (NGS)-based assay.

In some embodiments of a method disclosed herein, the diagnostic indicator results from a fluorescence in situ hybridization (FISH) assay.

In some embodiments of a method disclosed herein, the diagnostic indicator comprises an indicator for ecDNA-derived oncogene amplification.

In some embodiments of a method disclosed herein, the diagnostic indicator is obtained from tumor or liquid biopsy.

In some embodiments of a method disclosed herein, the method further comprises assessing a sample from a subject for the presence or level of one or more of a gene amplification, a focal gene amplification, ecDNA, HSR, or an ecDNA signature.

In some embodiments of a method disclosed herein, the method further comprises obtaining information of the presence or level of one or more of a gene amplification, a focal gene amplification, ecDNA, HSR, or an ecDNA signature in the tumor or tumor cells from the subject prior to, during or subsequent to the administration of Compound 1, or a pharmaceutically acceptable salt thereof.

### Oncogene Amplification

Oncogene amplification-associated tumors are a segment of the cancer population with an extremely high unmet need. Patients whose cancers harbor high-copy oncogene amplification have significantly worse survival compared with the broader cancer population. Pan-cancer analysis of oncogene-amplified tumors, cross-referenced with the Surveillance, Epidemiology, and End Results program data, indicates that, in the US alone, this population represents more than 400,000 newly diagnosed cancer patients each year across multiple tumor types.

Despite the enormous health benefits and improvements in survival afforded by precision medicine and targeted therapies for cancer, these therapies have unfortunately proven largely non-efficacious in the oncogene amplification population. In addition, immune checkpoint inhibitors (e.g., pembrolizumab) might perform poorly in oncogene-amplified cancer populations and hyper-progression has been associated with oncogene-amplified tumor settings.

To date, HER2 inhibitors (e.g., trastuzumab) for HER2-overexpressing breast cancer, gastroesophageal junction cancer, and gastric cancer are the only targeted therapies approved in oncogene amplified (or overexpressed) cancer populations, with breast cancer being the only single agent approval. Targeted agents that have shown efficacy in patients whose cancers are driven by oncogene point mutations, gene fusions or skipping deletions have generally failed to demonstrate robust efficacy in patients whose tumors are driven by oncogene amplification. This lack of approved therapies is despite extensive clinical testing of targeted agents in oncogene-amplified cancer populations including EGFR inhibitors in EGFR-amplified glioblastoma multiforme, FGFR inhibitors in FGFR-amplified cancers, and CDK4/6 inhibitors in CDK4-amplified liposarcoma. These clinical data have resulted in the misconception that oncogene amplification may not be a relevant cancer driver. This erroneous conclusion is despite extensive data to the contrary. Recurrent focal copy number amplification and overexpression of established oncogene drivers (otherwise activated by mutation and/or gene fusion), as well as antitumor efficacy of targeted inhibition (genetic and pharmacologic) in short-term preclinical cancer models establishes that amplifications are drivers, even if targeted therapy treatment approaches are generally not translating to prolonged clinical benefit. However, the above disconnect suggests that cancers driven by oncogene amplifications are biologically different from other tumors and require a new therapeutic paradigm. Accordingly, improved understanding of oncogene amplification biology is necessary, with the objectives of advancing new therapeutic approaches, pharmaceutical targets, and new molecular entities for this high unmet need patient population.

### Role of Extrachromosomal DNA in Oncogene Amplification

Chromosomal instability and tumor heterogeneity have been suggested to account for many targeted therapy failures. Consistent with this hypothesis, oncogene amplification is a consequence of prior or ongoing chromosomal instability arising through either numerical and/or structural alterations in chromosomes and can give rise to ecDNA. It has long been recognized that oncogenes can be amplified not only on chromosomes but also on ecDNA, originally referred to as “double minutes”. However, the frequency, importance, and specific roles of ecDNA in cancer biology have not been well understood until recently.

Some of the most prevalent driver oncogenes are encoded on ecDNA and can confer a selective advantage to cancer cells. These oncogenes amplified on ecDNA have several features that distinguish them from chromosomally localized oncogene amplifications:
1. ecDNA lack centromeres, thus, in contrast to chromosomally localized amplification states, they segregate unequally into daughter cells during cell division. This property supports a non-Mendelian inheritance pattern, enabling extreme gene copy number changes in relatively few cell divisions and leads to extensive copy number heterogeneity driving adaptability and tumor evolution.
2. ecDNA are epigenetically dysregulated and contain accessible chromatin and hyper-transcribed gene regions that are often more actively expressed than chromosomally located genes.

These features distinguish ecDNA from other forms of oncogene amplification and facilitate a level of genomic plasticity and adaptability beyond chromosomal amplification that enable tumors to evade environmental insults, including targeted therapeutic pressure. New therapeutic approaches that interfere with ecDNA function are necessary to overcome ecDNA mediated adaptation in oncogene-amplified tumors.

ecDNA-enabled oncogene amplifications are a primary driver of oncogenesis, play a critical role in driving tumor heterogeneity, and enable cancer cells to rapidly become resistant to targeted oncogene therapies. ecDNA-enabled oncogene amplifications were observed in nearly half of all human cancer types but almost never found in normal cell. For example, ecDNA-enabled oncogene amplifications can be found in approximately 14% of primary cancer specimens and that more than half of all high-copy number oncogene amplifications (i.e., copy number value >8) reside on ecDNA. Further, many of the most aggressive tumor types contain the highest prevalence of ecDNA, including approximately 60% of glioblastoma multiforme and just under 50% of sarcomas.

Patients whose cancers harbor ecDNA experience significantly shorter survival than cancer patients whose tumors are driven by other molecular lesions, even when controlled for tumor type. These data strongly indicate that patients with ecDNA-enabled cancers require a new therapeutic paradigm to address this large unmet need.

### Role of Extrachromosomal DNA in Therapeutic Resistance

The unique features of ecDNA, coupled with the remarkable genome plasticity of ecDNA-enabled tumors, contribute to the tumor's aggressive nature and ability to evade therapeutic pressure via rapid genomic evolution. The first demonstration of therapeutic resistance driven by ecDNA was in a mouse cancer cell line whereby methotrexate treatment led to high amplification of dihydrofolate reductase (DHFR) on ecDNA, and which was lost upon removal of methotrexate. Similar instances of DHFR ecDNA amplification have been recapitulated in multiple human cancer cell lines. Furthermore, amplification of drug efflux pump genes on ecDNA, including the family of ABC transporters, has been observed to mediate resistance to various chemotherapies. An equivalent role for ecDNA in providing resistance to more current targeted therapies has also been well established. Evasion of therapeutic response to the EGFR inhibitor erlotinib is facilitated by rapid loss of the population of EGFRvIII amplifications on ecDNA in patient-derived glioblastoma multiforme cells, contemporaneous with occurrence of a new cell population containing MDM2 amplification on ecDNA; this observed effect is consistent with an equivalent lack of response to EGFR inhibitors in patients. Preclinical studies in a gastric cancer cell line containing FGFR2 amplified on ecDNA demonstrated that cellular resistance to the pan-FGFR inhibitor infigratinib could be driven by oncogene dependency switching from FGFR2 amplification on ecDNA to a new, rapid amplification of EGFR on ecDNA. Strikingly, this dependency was reversed back to FGFR2 amplification on ecDNA under EGFR inhibitory pressure via erlotinib. In each case, the initial cell population was sensitive to the respective targeted therapy, resulting in short lived anti-proliferative effects lasting several weeks. Resistance and regrowth to the targeted therapies occurred coincident with switching of the amplified oncogenes on ecDNA. The rapid rate of amplification change is unique to ecDNA and helps account for the intrinsic targeted therapy resistance of de novo oncogene-amplified cancers.

Similarly, mutant oncogenes (e.g., BRAFV600E and KRASG12C) can be amplified on ecDNA as a resistance mechanism to corresponding targeted therapies (e.g., BRAF/MEK or KRAS inhibitors). For example, a mutant BRAFV600E melanoma cell line developed ecDNA-enabled amplification of BRAFV600E after exposure to BRAF/MEK dual inhibition. This phenomenon has also been documented in clinical cases. Relatedly, numerous putative acquired resistance mechanisms to the KRASG12C inhibitor adagrasib have been reported and of these, a high-level focal amplification of KRASG12C on ecDNA, confirmed in vitro and in vivo, conferred resistance to both clinically validated KRASG12C inhibitors, adagrasib and sotorasib.

Collectively, these studies highlight the striking genomic plasticity and precipitous rise of ecDNA enabled oncogene amplification that enables cancer cells to adapt rapidly to therapeutic pressure. In conclusion, cancers driven by ecDNA-enabled oncogene amplification are biologically different from other oncogene activated tumors and require a new therapeutic paradigm.

### Dosing/Administration

Disclosed herein is a method of treating cancer in a subject in need thereof comprising administering to the subject a pharmaceutical composition comprising Compound 1, or a pharmaceutically acceptable salt thereof. In some embodiments, the pharmaceutical composition comprising Compound 1, or a pharmaceutically acceptable salt thereof is suitable for oral administration and such composition is formulated as an orally available pharmaceutical composition as described herein. In some cases, the pharmaceutical composition comprising Compound 1, or a pharmaceutically acceptable salt thereof is formulated as a tablet for oral administration.

### Rationale for Human Starting Dose of Compound 1

GLP-compliant repeat dose toxicology studies were conducted in rats and dogs following oral gavage Q2D with exposure duration of up to 29 days. The main effects observed in both species were related to bone marrow suppression/depletion and gastrointestinal toxicity, and were considered an on-target pharmacological effect of Compound 1 that is directly associated with CHK1 inhibition. Partial or complete reversibility of Compound 1-related major changes was demonstrated in all tissues, and there was no major unexpected toxicity identified.

In some embodiments of a method disclosed herein, Compound 1 is administered at a dose of about 10 mg to about 800 mg.

In some embodiments of a method disclosed herein, Compound 1 is administered at a dose of about 10 mg to about 400 mg.

In some embodiments of a method disclosed herein, Compound 1 is administered at a dose of about 10 mg to about 400 mg, whereby the subject experiences a therapeutic response.

In some embodiments of a method disclosed herein, the dose of compound 1 is between about 10 mg to about 20 mg, about 20 mg to about 40 mg, about 40 mg to about 80 mg, about 80 mg to about 120 mg, about 120 mg to about 160 mg, about 160 mg to about 200 mg, about 200 mg to about 400 mg.

In some embodiments of a method disclosed herein, the dose of compound 1 is between about 20 to about 40 mg, about 40 to about 80 mg, about 80 to about 120 mg, about 120 mg to about 160 mg, or about 160 mg to about 200 mg.

In some embodiments of a method disclosed herein, the dose of compound 1 is between about 10 mg to about 20 mg, about 20 mg to about 40 mg, about 40 to about 80 mg, or about 80 to about 120 mg.

In some embodiments of a method disclosed herein, the dose of compound 1 is between about 10 mg to about 20 mg. In some embodiments of a method disclosed herein, the dose of compound 1 is between about 20 mg to about 40 mg. In some embodiments of a method disclosed herein, the dose of compound 1 is between about 40 mg to about 80 mg. In some embodiments of a method disclosed herein, the dose of compound 1 is between about 80 mg to about 120 mg. In some embodiments of a method disclosed herein, the dose of compound 1 is between about 120 mg to about 160 mg. In some embodiments of a method disclosed herein, the dose of compound 1 is between about 160 mg to about 200 mg. In some embodiments of a method disclosed herein, the dose of compound 1 is between about 200 mg to about 400 mg.

In some embodiments of a method disclosed herein, the dose of compound 1 is about 10 mg, about 20 mg, about 40 mg, about 80 mg, about 120 mg, about 160 mg, or about 200 mg. In some embodiments of a method disclosed herein, the dose of compound 1 is about 10 mg. In some embodiments of a method disclosed herein, the dose of compound 1 is about 15 mg. In some embodiments of a method disclosed herein, the dose of compound 1 is about 20 mg. In some embodiments of a method disclosed herein, the dose of compound 1 is about 25 mg. In some embodiments of a method disclosed herein, the dose of compound 1 is about 30 mg. In some embodiments of a method disclosed herein, the dose of compound 1 is about 35 mg. In some embodiments of a method disclosed herein, the dose of compound 1 is about 40 mg. In some embodiments of a method disclosed herein, the dose of compound 1 is about 45 mg. In some embodiments of a method disclosed herein, the dose of compound 1 is about 50 mg. In some embodiments of a method disclosed herein, the dose of compound 1 is about 55 mg. In some embodiments of a method disclosed herein, the dose of compound 1 is about 60 mg. In some embodiments of a method disclosed herein, the dose of compound 1 is about 65 mg. In some embodiments of a method disclosed herein, the dose of compound 1 is about 70 mg. In some embodiments of a method disclosed herein, the dose of compound 1 is about 75 mg. In some embodiments of a method disclosed herein, the dose of compound 1 is about 80 mg. In some embodiments of a method disclosed herein, the dose of compound 1 is about 85 mg. In some embodiments of a method disclosed herein, the dose of compound 1 is about 90 mg.

In some embodiments of a method disclosed herein, the dose of compound 1 is about 95 mg. In some embodiments of a method disclosed herein, the dose of compound 1 is about 100 mg. In some embodiments of a method disclosed herein, the dose of compound 1 is about 105 mg. In some embodiments of a method disclosed herein, the dose of compound 1 is about 110 mg. In some embodiments of a method disclosed herein, the dose of compound 1 is about 115 mg. In some embodiments of a method disclosed herein, the dose of compound 1 is about 120 mg. In some embodiments of a method disclosed herein, the dose of compound 1 is about 125 mg. In some embodiments of a method disclosed herein, the dose of compound 1 is about 130 mg. In some embodiments of a method disclosed herein, the dose of compound 1 is about 135 mg. In some embodiments of a method disclosed herein, the dose of compound 1 is about 140 mg. In some embodiments of a method disclosed herein, the dose of compound 1 is about 145 mg. In some embodiments of a method disclosed herein, the dose of compound 1 is about 150 mg. In some embodiments of a method disclosed herein, the dose of compound 1 is about 155 mg. In some embodiments of a method disclosed herein, the dose of compound 1 is about 160 mg. In some embodiments of a method disclosed herein, the dose of compound 1 is about 165 mg. In some embodiments of a method disclosed herein, the dose of compound 1 is about 170 mg. In some embodiments of a method disclosed herein, the dose of compound 1 is about 175 mg. In some embodiments of a method disclosed herein, the dose of compound 1 is about 180 mg. In some embodiments of a method disclosed herein, the dose of compound 1 is about 185 mg. In some embodiments of a method disclosed herein, the dose of compound 1 is about 190 mg. In some embodiments of a method disclosed herein, the dose of compound 1 is about 195 mg. In some embodiments of a method disclosed herein, the dose of compound 1 is about 200 mg.

In some embodiments of a method disclosed herein, the composition is administered orally.

In some embodiments of a method disclosed herein, the composition is administered parentally.

In some embodiments of a method disclosed herein, the composition is administered every day.

In some embodiments of a method disclosed herein, the composition is administered every other day.

In some embodiments of a method disclosed herein, the composition is administered on a cycle of day 1 and day 3 followed by a 4 day dosing holiday.

In some embodiments of a method disclosed herein, the composition is administered every 3 days or weekly.

In some embodiments of a method disclosed herein, the composition is administered every 3 days. In some embodiments of a method disclosed herein, the composition is administered weekly.

In some embodiments of a method disclosed herein, the composition is administered with a dosing holiday of 4 days, 4-7 days, 7 days, or 14 days.

### Combination

Disclosed herein is a method of treating cancer in a subject in need thereof comprising administering to the subject a pharmaceutical composition comprising a Compound 1, or a pharmaceutically acceptable salt thereof and an additional therapeutic agent. In some embodiments, the CHK1 inhibitor is Compound 1, or a pharmaceutically acceptable salt thereof. In some embodiments, the pharmaceutical composition comprising Compound 1, or a pharmaceutically acceptable salt thereof is suitable for oral administration and such composition is formulated as an orally available pharmaceutical composition as described herein. In some cases, the pharmaceutical composition comprising Compound 1, or a pharmaceutically acceptable salt thereof is formulated as a tablet for oral administration. In some cases, the pharmaceutical composition comprising Compound 1, or a pharmaceutically acceptable salt thereof is for oral administration and the additional therapeutic agent also is formulated for oral administration.

In some embodiments of a method disclosed herein, the additional therapeutic agent is an EGFR inhibitor.

In some embodiments of a method disclosed herein, the EGFR inhibitor is selected from the group consisting of 602, 705, 707, abivertinib, ABX-900, afatinib, agerafenib (RXDX-105), alflutinib mesylate, amivantamab, APL-1898, ASK-120067, aumolertinib (almonertinib), BBT-176, BDTX-1535, BDTX-189, BEBT-109, befortinib mesylate, beitatini, BLU-701, BLU-945, BPI-361175, BPI-7711, BPI-D0316, C-005, CDP1, cetuximab, CH-7233163, CK-101, CMAB-017, dacomitinib, depatuxizumab, depatuxizumab mafodotin (ABT-414), DFP-17729, dositinib, DS-2087, DZD-9008, E01001, E-10C, epertinib, epitinib (HMPL-813), erlotinib, ES-072, FCN-411, FHND-9041, furmonertinib, FWD-1509, GB-263, GC-1118A, gefitinib, GMA-204, GR-1401, Hemay-022, HLX-07, HS-627, I-010, icotinib, imgatuzumab, IN-A008, JMT-101, JRF-103, JS-111, JS-113, JZB-28, KN-023, KN-026, KP-673, lapatinib, larotinib, lazertinib, LL-191, LYN 205, M1231, maihuatinib, marizomib, mobocertinib, MP-0274, MRG003, naputinib tosilate, nazartinib, necitumumab, neptinib, nimotuzumab, NRC-2694-A, NT-004, OBX1-012, olafertinib, olmutinib, ORIC-114, oritinib, osimertinib, panitumumab, pirotinib, poziotinib, PRB-001, pyrotinib, QL-1203, SCT-200, serclutamab, SHR-A1307, SIM-200, SPH-1188, SSGJ-612, SYN-004, TAD-011, tarloxotinib, TAS-6417, TGRX-360, theliatinib (HMPL-309), TPC-064, TQB-3804, TY-9591, WJ-13404, WSD-0922, XZP-5809, yinlitinib maleate, YK-029A, YZJ-0318, zorifertinib, and ZSP-0391.

In some embodiments of a method disclosed herein, the EGFR inhibitor is erlotinib. Erlotinib (TARCEVA®) is an oral small molecule inhibitor of the receptor tyrosine kinase EGFR. Early data showed anticancer activity in a several tumors including cancers of the lung and pancreas, and erlotinib was approved by the FDA in 2013 for EGFR mutant NSCLC with EGFR exon 19 deletions or exon 21 substitution mutations. Notably, erlotinib can inhibit wildtype EGFR and is not selective for mutant EGFR only.

The planned dosage of erlotinib is 150 mg orally once daily, given >1 hour before or 2 hours after food intake. This is the dose of erlotinib for treatment of NSCLC per the TARCEVA® United States Prescribing Information (USPI).

In some embodiments of a method disclosed herein, erlotinib is administered to the subject at a dose of 150 mg PO daily, 100 mg PO daily, or 50 mg PO daily.

In some embodiments of a method disclosed herein, the additional therapeutic agent is a FGFR inhibitor.

In some embodiments of a method disclosed herein, the FGFR inhibitor is selected from the group consisting of 3D-185, ABSK-011, ABSK-012, ABSK-061, ABSK-091, aldafermin, alofanib, AST-56100, AZD-4547, bemarituzumab, BFKB-8488A, BGS-2219, BIO-1262, BPI-17509, BPI-43487, CPL-304-110, derazantinib, E-7090, erdafitinib, EVER-4010001, EVT-601, FGF-401, fisogatinib, FPI-1966, futibatinib, gunagratinib, H3B-6527, HH-185, HMPL-453, HS-236, ICP-105, ICP-192, infigratinib, JAB-6000, KIN-3248, M-6123, MAX-40279, OM-RCA-001, pemigatinib, RLY-4008, rogaratinib, SAR-439115, SAR-442501, SC-0011, SY-4798, TT-00434, zoligratinib (FF-284), and WXSH-0011.

In some embodiments of a method disclosed herein, the FGFR inhibitor is pemigatinib. Pemigatinib (PEMAZYRE®) is an oral small molecule inhibitor of the receptor tyrosine kinase FGFR. Pemigatinib was first approved by the FDA in 2020 for previously treated, unresectable locally advanced or metastatic cholangiocarcinoma with a FGFR2 fusion or other rearrangement as detected by an FDA-approved test. Notably, pemigatinib can inhibit wildtype FGFR1, FGFR2, and FGFR3 receptors.

In some embodiments of a method disclosed herein, pemigatinib is administered to the subject at a dose of 13.5 mg PO daily, 9 PO mg, 4.5 mg PO daily and the dose is administered once daily for 14 days followed by 7 sequential days without administration of pemigatinib.

In some embodiments of a method disclosed herein, the FGFR inhibitor is futibatinib.

Futibatinib (LYTGOBIR) is indicated for the treatment of adults with previously treated, unresectable, locally advanced, or metastatic intrahepatic cholangiocarcinoma harboring fibroblast growth factor receptor 2 (FGFR2) gene fusions or other rearrangements. Futibatinib was approved for medical use in the United States in September 2022.

In some embodiments of a method disclosed herein, futibatinib is administered to the subject at a dose of 20 mg PO daily.

In some embodiments of a method disclosed herein, the additional therapeutic agent is a CDK4/6 inhibitor.

In some embodiments of a method disclosed herein, the CDK4/6 inhibitor is selected from the group consisting of abemaciclib, AG-122275, AM-5992, AT-7519, AU2-94, auceliciclib, BEBT-209, BPI-1178, BPI-16350, CS-3002, fascaplysin, FCN-437, FN-1501, GLR-2007, GW-491619, HEC-80797, HS-10342, IIIM-290, IIIM-985, lerociclib, milciclib maleate, MM-D37K, MS-140, NP-102, NUV-422, ON-123300, palbociclib, PF-06842874, PF-06873600, PF-07220060, QHRD-110, R-547, RGB-286199, RGT-419B, ribociclib, riviciclib, RO-0505124, SHR-6390, THR-53, THR-79, TQB-3303, TQB-3616, trilaciclib, TY-302, TY-302, voruciclib, VS2-370, WXWH-0240, XH-30002, and XZP-3287.

In some embodiments of a method disclosed herein, the CDK4/6 inhibitor is abemaciclib. Abemaciclib (VERZENIO®) is an oral small molecule inhibitor of CDK4/6. Abemaciclib was first approved by the FDA in 2017 for advanced or metastatic breast cancer, which is hormone receptor positive and HER-2 negative. Notably, abemaciclib can inhibit wildtype CDK4 and CDK6 receptors.

In some embodiments of a method disclosed herein, the abemaciclib is administered to the subject at a dose of about 50 mg twice daily, about 100 mg twice daily, or 150 mg twice daily.

In some embodiments of a method disclosed herein, the additional therapeutic agent is a BRAF inhibitor. In some embodiments of a method disclosed herein, the BRAF inhibitor is ABM-1310, agerafenib (RXDX-105), ARQ-736, ASN-003, AZ-304, AZ-628, BAL-3833, belvarafenib, BGB-3245, BI-882370, dabrafenib, DAY101, DP-2874, EBI-907, EBI-945, encorafenib, GDC-0879, lifirafenib, LUT-014, LYN 204, NMS-P285, NMS-P730, PF-04880594, PF-07284890, PLX-8394, RX-208, TL-241, UAI-201, UB-941, vemurafenib, VS-6766, or XL-281.

In some embodiments of a method disclosed herein, the additional therapeutic agent is a MDM2 or MDM4 inhibitor.

In some embodiments of a method disclosed herein, the MDM2 inhibitor is AD-021.32, ALRN-6924, APG-115, ASTX-295, ATSP-7041, BI-907828, CGM-097, CYC700, DS-5272, idasanutlin, KRT-232 (AMG-232), MD-224, MI-1061, MI-219, MI-43, MI-77301 (SAR405838, SAR299155), MK-8242, NU-8231, NVP-CGM097, OM-301, PXN-527, RAIN-32 (milademetan), RG7112 (RO5045337), RG7388 (RG7775), Rigel-3, RO-2468, RO-5353, RO-5963, serdemetan (JNJ-26854165), SIL-43, siremadlin, or UBX-0101. In some embodiments of a method disclosed herein, the MDM4 inhibitor is17AAG, 489-PXN, ALRN-6924, APG-115, ATSP-7041, BI-907828, CTX1, FL-118, inulanolide A, K-178, or SAH-p53-8.

In some embodiments of a method disclosed herein, the additional therapeutic agent is a MET inhibitor.

In some embodiments of a method disclosed herein, the MET inhibitor is ABP-1130, BPI-1831, BPI-2021, BYON-3521, CG-203306, CX-1003, Debio-1144, EMD-94283, EMT-100, EMT-101, HE-003, LMV-12, LS-177, NX-125, OMO-2, PF-4254644, PRX-MET, PTX-2173, QBH-196, RP-1400, SAB-Y14, SAR-125844, SGX-126, SYD-3521, WXSH-0011, X-379, and XL-265, and anti-MET antibodies such as ABX-900, GB-263, FS-101, LY-3164530, LY-3343544, PMC-002, or SAIT-301. In some embodiments of a method disclosed herein, the MET inhibitor is ABN-401, ABT-700, AMG-208, AMG-337, ARGX-111, BAY-85-3474, BMS-817378, bozitinib, BPI-9016M, glumetinib, golvatinib tartrate, GST-HG161, HQP-8361, I-020, JNJ-38877605, kanitinib, merestinib, MK-2461, MK-8033, OMO-1, pamufetinib, S-49076, savolitinib, SPH-3348, tivantinib, SAR-125844, SCR-1515, and TPX-0022, or anti-MET antibodies such as APL-101, CKD-702, EMB-01, EMI-137, ficlatuzumab, HLX-55, HS-10241, MCLA-129, MT-8633 NOV-1105, RC-108, REGN-5093, SHR-A1403, Sym-015, or telisotuzumab vedotin. In some embodiments of a method disclosed herein, the MET inhibitor is amivantamab, capmatinib, crizotinib, or tepotinib.

In some embodiments of a method disclosed herein, the additional therapeutic agent is a KRAS inhibitor. In some embodiments of a method disclosed herein, the KRAS inhibitor is ABREV01, ARS-1620, APG-1842, ATG-012, BBP-454, BEPT-607, BI-2852, BI-1823911, BPI-421286, BTX-2541, COTI-219, IMM-1811900, JAB-21000, JAB-22000, JAB-23000, JAB-BX300, JP-002, KR-12, LYN 202, MRTX-1133, RAS-F, RMC-6236, RMC-6291, SDGR 5, STX-301, and YL-15293, or anti-KRAS antibodies such as SBT-100, SBT-102, or SBT-300. In some embodiments of a method disclosed herein, the KRAS inhibitor is adagrasib, ARS-3248, D-1553, GDC-6036, JDQ-443, LY3537982, sotorasib (AMG 510), or BI 1701963.

### EXAMPLES

### Example 1

The quantitative compositions of representative Compound 1 tablets are presented in Table 1.

TABLE 1
Quantitative compositions of Compound 1 tablets, 5 mg and 20 mg

| | | | Quantity per Tablet (mg) | | |
|---|---|---|---|---|---|
| | Quality | | | 5 mg | 20 mg |
| Component | Standard | Function | % | Tablet | Tablet |
| Compound 1 | In-house | API | 10.0 | 5.0 | 20.0 |
| Microcrystalline | NF, Ph. | Diluent | 85.0 | 42.5 | 170.0 |
| Cellulose | Eur | | | | |
| Croscarmellose | NF, Ph. | Disintegrant | 3.0 | 1.5 | 6.0 |
| Sodium | Eur | | | | |
| Colloidal Silicon | NF, Ph. | Glidant | 1.0 | 0.5 | 2.0 |
| Dioxide | Eur | | | | |
| Magnesium | NF, Ph. | Lubricant | 1.0 | 0.5 | 2.0 |
| Stearate | Eur | | | | |

| | | | |
|---|---|---|---|
| Core Tablet | 100.0 | 50.0 | 200.0 |

Film Coating

| | | | | | |
|---|---|---|---|---|---|
| Opadry | Proprietary | Film | 3.0 | 1.5 | 6.0 |
| 03K120035 | | coating | | | |
| Yellow | | | | | |

| | | | |
|---|---|---|---|
| Purified Water | USP | Solvent | ^{d} |

| | | | |
|---|---|---|---|
| Film Coated Tablet | 103.0 | 51.5 | 206.0 |

API = active pharmaceutical ingredient;
NF = National Formulary;
Ph. Eur = European Pharmacopeia;
USP = United States Pharmacopeia
^{d} Removed during processing

Opadry 03K120035 yellow, supplied by Colorcon Inc, is not a compendial excipient; but the sub-constituents are compendial. The qualitative composition of the film coating material, based on technical product documentation from Colorcon Inc, is provided in Table 2.

TABLE 2
Qualitative compositions of the Opadry 03K120035 Yellow System

| | Quality | |
|---|---|---|
| Component | Standard | Function |
| Hypromellose | NF, Ph. Eur | Film former |
| Triacetin | NF, Ph. Eur | Plasticizer |
| Titanium Dioxide | NF, Ph. Eur | Opacifying agent |
| Yellow Iron Oxide | NF | Colorant |

NF = Nation Formulary;
Ph.Eur = European Pharmacopeia;
USP = United States Pharmacopeia

### Example 2: Stability

Stability studies were initiated for the development batches at long term and accelerated storage conditions. Stability tests include appearance, assay, impurities, water content and dissolution.

After 2 months at 25° C./60% RH and 40° C./75% RH storage conditions, both R&D batches showed no significant trends in appearance, assay, and dissolution. Trends of increased impurities and decreased water content were observed.

As a result, stability studies for scale-up batches were initiated at 2-8° C. long-term and 25° C./60% RH accelerated stability conditions. Initial 1-month data at both storage conditions indicate no apparent trends in appearance, assay, purity, water content and dissolution.

### Stability Data

Details of drug product batches currently in storage stability studies are provided in Tables 3 and Table 4 Storage stability data are provided in Tables 5-12.

TABLE 3
Details of Compound 1 Tablets, 5 mg in Stability Studies

| Batch Number | #1 | #2 |
|---|---|---|
| Batch size (kg) | 0.27¹ | 1.6 |
| Use | R&D | Scale-up |
| | Stability | stability |
| Stability data, | 2 months | NA |
| 40° C./75% RH | | |
| Stability data, | 2 months | 1 month |
| 25° C./60% RH | | |
| Stability data, | NA | 1 month |
| 2-8° C. | | |

¹Common blend

TABLE 4
Details of Compound 1 Tablets, 20 mg in Stability Studies

| Batch Number | #3 | #4 |
|---|---|---|
| Batch size (kg) | 0.27¹ | 3.0 |
| Use | R&D | Scale-up |
| | Stability | stability |
| Stability data, | 2 months | NA |
| 40° C./75% RH | | |
| Stability data, | 2 months | 1 month |
| 25° C./60% RH | | |
| Stability data, | NA | 1 month |
| 2-8° C. | | |

¹Common blend

TABLE 5
Stability Data for R&D Stability Batch Compound 1, 5 mg
Tablets, Batch #1, stored at 25° C./60% RH
Batch No.: #1
Storage condition: 25° C./60% RH
Container closure: 15 count in HDPE bottle with CRC/PP

| | Accep- | | | | | | | |
|---|---|---|---|---|---|---|---|---|
| | tance | Time (Months) | | | | | | |
| Test | Criteria | Initial | | 1 | 2 | | 3 | 6 |
| Description | Report | A | | NT | A | | — | — |
| Assay | Report | 97.2 | | 96.0 | 95.7 | | — | — |
| (% LC) | | | | | | | | |
| Water | Report | 4.7 | | 3.1 | 3.4 | | — | — |
| content | | | | | | | | |
| (% w/w) | | | | | | | | |
| Individual | | | | | | | | |
| impurities | | | | | | | | |
| (% a/a)¹ | | | | | | | | |
| RRT 0.85 | Report | ND | | ND | 0.08 | | — | — |
| RRT 0.87 | | 0.35 | | 0.35 | 0.07 | | — | — |
| RRT 0.90 | | ND | | ND | 0.21 | | — | — |
| RRT 1.04 | | 0.84 | | 1.07 | 1.44 | | — | — |
| RRT 1.08 | | ND | | ND | ND | | — | — |
| RRT 1.11 | | ND | | ND | 0.04 | | — | — |
| Total | Report | 1.2 | | 1.4 | 1.8 | | — | — |
| impurities | | | | | | | | |
| (% a/a) | | | | | | | | |
| | | | Ave | | | Ave | | |
| Dissolution | Report | 10 min | 88 | NT | 10 min | 92 | — | — |
| (profile, | | 15 min | 94 | | 15 min | 97 | | |
| ave % | | 20 min | 97 | | 20 min | 99 | | |
| dissolved) | | 30 min | 99 | | 30 min | 99 | | |
| | | 45 min | 99 | | 45 min | 99 | | |
| | | 60 min | 99 | | 60 min | 99 | | |

A = Pale yellow to yellow colored, round, biconvex tablets;
NT = Not tested;
HDPE = high density polyethylene;
PP = polypropylene;
CRC: Child Resistant Cap;
¹approximate RRTs,
RRT = relative retention time;
RH = relative humidity;
— scheduled testing

TABLE 6
Stability Data for R&D Stability Batch Compound 1, 5 mg
Tablets, Batch #1, stored at 40° C./75% RH
Batch No.: #1
Storage condition: 40° C./75% RH
Container closure: 15 count in HDPE bottle with CRC/PP

| | Acceptance | Time (Months) | | | | | | | |
|---|---|---|---|---|---|---|---|---|---|
| Test | Criteria | Initial | | 1 | | 2 | | 3 | 6 |
| Description | Report | A | | NT | | A | | — | — |
| Assay (% LC) | Report | 97.2 | | 97.2 | | 96.5 | | — | — |
| Water content | Report | 4.7 | | 3.3 | | 3.6 | | — | — |
| (% w/w) | | | | | | | | | |
| IndIndividual | | | | | | | | | |
| impurities (% a/a) | | | | | | | | | |
| RRT 0.85 | Report | ND | | ND | | 0.05 | | — | — |
| RRT 0.87 | | 0.35 | | 0.33 | | 0.05 | | — | — |
| RRT 0.90 | | ND | | ND | | 0.22 | | — | — |
| RRT 1.04 | | 0.84 | | 1.47 | | 1.78 | | — | — |
| RRT 1.08 | | ND | | 0.04 | | 0.10 | | — | — |
| RRT 1.11 | | ND | | ND | | 0.11 | | — | — |
| Total impurities | Report | 1.2 | | 1.8 | | 2.3 | | — | — |
| (% a/a) | | | | | | | | | |
| | | | Ave | | Ave | | Ave | | |
| Dissolution | Report | 10 min | 88 | 10 min | 95 | 10 min | 91 | — | — |
| (profile, ave % | | 15 min | 94 | 15 min | 98 | 15 min | 96 | | |
| dissolved) | | 20 min | 97 | 20 min | 100 | 20 min | 98 | | |
| | | 30 min | 99 | 30 min | 100 | 30 min | 99 | | |
| | | 45 min | 99 | 45 min | 100 | 45 min | 99 | | |
| | | 60 min | 99 | 60 min | 100 | 60 min | 99 | | |

A = Pale yellow to yellow colored, round, biconvex tablets;
NT = Not tested;
HDPE = high density polyethylene;
PP = polypropylene;
CRC: Child Resistant Cap
¹ approximate RRTs,
RRT = relative retention time;
RH = relative humidity;
— scheduled testing

TABLE 7
Stability Data for R&D Stability Batch Compound 1, 20 mg
Tablets, Batch #3, at 25° C./60% RH
Batch No.: #3
Storage condition: 25° C./60% RH
Container closure: 15 count in HDPE bottle with CRC/PP

| | Accep- | | | | | | | |
|---|---|---|---|---|---|---|---|---|
| | tance | Time (Months) | | | | | | |
| Test | Criteria | Initial | | 1 | 2 | | 3 | 6 |
| Description | Report | A | | NT | A | | — | — |
| Assay | Report | 99.3 | | 96.3 | 98.7 | | — | — |
| (% LC) | | | | | | | | |
| Water | Report | 5.0 | | 3.8 | 3.9 | | — | — |
| content | | | | | | | | |
| (% w/w) | | | | | | | | |
| Individual | | | | | | | | |
| impurities | | | | | | | | |
| (% a/a) | | | | | | | | |
| RRT 0.85 | Report | ND | | ND | 0.07 | | — | — |
| RRT 0.87 | | 0.34 | | 0.34 | 0.07 | | — | — |
| RRT 0.90 | | ND | | ND | 0.20 | | — | — |
| RRT 1.04 | | 0.74 | | 1.01 | 1.34 | | — | — |
| RRT 1.08 | | ND | | ND | ND | | — | — |
| RRT 1.11 | | ND | | ND | 0.04 | | — | — |
| Total | Report | 1.1 | | 1.4 | 1.7 | | — | — |
| impurities | | | | | | | | |
| (% a/a) | | | | | | | | |
| | | | Ave | | | Ave | | |
| Dissolution | Report | 10 min | 79 | NT | 10 min | 90 | — | — |
| (profile, | | 15 min | 84 | | 15 min | 93 | | |
| ave % | | 20 min | 87 | | 20 min | 95 | | |
| dissolved) | | 30 min | 90 | | 30 min | 95 | | |
| | | 45 min | 93 | | 45 min | 95 | | |
| | | 60 min | 100 | | 60 min | 95 | | |

A = Pale yellow to yellow colored, round, biconvex tablets;
NT = Not tested;
HDPE = high density polyethylene;
PP = polypropylene;
CRC: Child Resistant Cap
¹ approximate RRTs,
RRT = relative retention time;
RH = relative humidity;
— scheduled testing

TABLE 8
Stability Data for R&D Stability Batch Compound 1, 20 mg
Tablets, Batch #3, at 40° C./75% RH
Batch No.: #3
Storage condition: 40° C./75% RH
Container closure: 15 count in HDPE bottle w CRC/PP

| | Acceptance | Time (Months) | | | | | | | |
|---|---|---|---|---|---|---|---|---|---|
| Test | Criteria | Initial | | 1 | | 2 | | 3 | 6 |
| Description | Report | A | | NT | | A | | — | — |
| Assay (% LC) | Report | 99.3 | | 95.3 | | 96.4 | | — | — |
| Water content | Report | 5.0 | | 4.0 | | 4.1 | | — | — |
| (% w/w) | | | | | | | | | |
| Individual | | | | | | | | | |
| impurities (% a/a) | | | | | | | | | |
| 0.85 | Report | ND | | ND | | 0.07 | | — | — |
| 0.87 | | 0.34 | | 0.30 | | 0.06 | | — | — |
| 0.90 | | ND | | ND | | 0.18 | | — | — |
| 1.04 | | 0.74 | | 1.36 | | 1.89 | | — | — |
| 1.08 | | ND | | ND | | 0.08 | | — | — |
| 1.11 | | ND | | ND | | 0.10 | | — | — |
| Total impurities | Report | 1.1 | | 1.7 | | 2.4 | | — | — |
| (% a/a) | | | | | | | | | |
| | | | Ave | | Ave | | Ave | | |
| Dissolution | Report | 10 min | 79 | 10 min | 91 | 10 min | 89 | — | — |
| (profile, ave % | | 15 min | 84 | 15 min | 94 | 15 min | 93 | | |
| dissolved) | | 20 min | 87 | 20 min | 95 | 20 min | 94 | | |
| | | 30 min | 90 | 30 min | 96 | 30 min | 95 | | |
| | | 45 min | 93 | 45 min | 96 | 45 min | 95 | | |
| | | 60 min | 100 | 60 min | 95 | 60 min | 94 | | |

A = Pale yellow to yellow colored, round, biconvex tablets;
NT = Not tested;
HDPE = high density polyethylene;
PP = polypropylene;
CRC: Child Resistant Cap
¹ approximate RRTs,
RRT = relative retention time;
RH = relative humidity;
— scheduled testing

TABLE 9
Stability Data for Scale-Up Batch Compound 1, 5 mg
Tablets, Batch #2, at 2-8° C.
Batch No.: #2
Storage condition: 2-8° C.
Container closure: 15 count in HDPE bottle with CRC/PP

| | Acceptance | Time (Months) | | | | | | | | | | | |
|---|---|---|---|---|---|---|---|---|---|---|---|---|---|
| Test | Criteria | Initial | | | | 1 | | | | 3 | 6 | 9 | 12 |
| Description | Report | A | | | | A | | | | — | — | — | — |
| Assay (% LC) | 90-110% | 96.8 | | | | 97.0 | | | | — | — | — | — |
| Water content | Report | 3.1 | | | | 3.5 | | | | — | — | — | — |
| (% w/w) | | | | | | | | | | | | | |
| Individual | | | | | | | | | | | | | |
| impurities (% a/a) | | | | | | | | | | | | | |
| RRT 0.90 | Report | 0.20 | | | | ND | | | | — | — | — | — |
| RRT 1.04 | | 1.27 | | | | 0.83 | | | | — | — | — | — |
| RRT 1.08 | | 0.10 | | | | 0.11 | | | | — | — | — | — |
| Total impurities | Report | 1.6 | | | | 0.94 | | | | — | — | — | — |
| (% a/a) | | | | | | | | | | | | | |
| | | | Min | Max | Ave | | Min | Max | Ave | | | | |
| Dissolution | Report | 10 | 93 | 98 | 95 | 10 | 89 | 101 | 97 | — | — | — | — |
| (profile, ave % | | 20 | 97 | 102 | 100 | 20 | 101 | 107 | 104 | | | | |
| dissolved) | | 30 | 97 | 102 | 100 | 30 | 102 | 107 | 104 | | | | |
| | | 45 | 98 | 103 | 100 | 45 | 101 | 107 | 104 | | | | |
| | | 60 | 97 | 102 | 100 | 60 | 102 | 107 | 104 | | | | |

A = Pale yellow to yellow colored, round, biconvex tablets;
HDPE = high density polyethylene;
PP = polypropylene;
CRC: Child Resistant Cap
¹ approximate RRTs,
RRT = relative retention time;
RH = relative humidity;
— scheduled testing

TABLE 10
Stability Data for Scale-Up Batch Compound 1, 5 mg
Tablets, Batch #2, at 25° C./60% RH
Batch No.: #2
Storage condition: 25° C./60% RH
Container closure: 15 count in HDPE bottle with CRC/PP

| | Acceptance | Time (Months) | | | | | | | | | | | |
|---|---|---|---|---|---|---|---|---|---|---|---|---|---|
| Test | Criteria | Initial | | | | 1 | | | | 3 | 6 | 9 | 12 |
| Description | Report | A | | | | A | | | | — | — | — | — |
| Assay (% LC) | 90-110% | 96.8 | | | | 98.6 | | | | — | — | — | — |
| Water content | Report | 3.1 | | | | 3.6 | | | | — | — | — | — |
| (% w/w) | | | | | | | | | | | | | |
| Individual | | | | | | | | | | | | | |
| impurities (% a/a) | | | | | | | | | | | | | |
| RRT 0.90 | Report | 0.20 | | | | ND | | | | — | — | — | — |
| RRT 1.04 | | 1.27 | | | | 0.92 | | | | — | — | — | — |
| RRT 1.08 | | 0.10 | | | | 0.111 | | | | — | — | — | — |
| Total impurities | Report | 1.6 | | | | 1.0 | | | | — | — | — | — |
| (% a/a) | | | | | | | | | | | | | |
| | | | Min | Max | Ave | | Min | Max | Ave | | | | |
| Dissolution (min, | Report | 10 | 93 | 98 | 95 | 10 | 95 | 102 | 98 | — | — | — | — |
| max, ave % | | 20 | 97 | 102 | 100 | 20 | 99 | 107 | 103 | | | | |
| dissolved) | | 30 | 97 | 102 | 100 | 30 | 100 | 108 | 103 | | | | |
| | | 45 | 98 | 103 | 100 | 45 | 99 | 108 | 103 | | | | |
| | | 60 | 97 | 102 | 100 | 60 | 98 | 108 | 103 | | | | |

A = Pale yellow to yellow colored, round, biconvex tablets;
HDPE = high density polyethylene;
PP = polypropylene;
CRC: Child Resistant Cap
¹ approximate RRTs,
RRT = relative retention time;
RH = relative humidity;
— scheduled testing

TABLE 11
Stability Data for Scale-Up Batch Compound 1 20 mg
Tablets, Batch #4, at 2-8° C.
Batch No.: #4
Storage condition: 2-8° C.
Container closure: 15 count in HDPE bottle with CRC/PP

| | Acceptance | Time (Months) | | | | | | | | | | | |
|---|---|---|---|---|---|---|---|---|---|---|---|---|---|
| Test | Criteria | Initial | | | | 1 | | | | 3 | 6 | 9 | 12 |
| Description | Report | A | | | | A | | | | — | — | — | — |
| Assay (% LC) | 90-110% | 99.1 | | | | 97.8 | | | | — | — | — | — |
| Water content | Report | 3.1 | | | | 3.3 | | | | — | — | — | — |
| (% w/w) | | | | | | | | | | | | | |
| Individual | | | | | | | | | | | | | |
| impurities (% a/a) | | | | | | | | | | | | | |
| RRT 0.85 | Report | 0.11 | | | | ND | | | | — | — | — | — |
| RRT 0.90 | | 0.18 | | | | ND | | | | — | — | — | — |
| RRT 1.04 | | 1.25 | | | | 0.83 | | | | — | — | — | — |
| RRT 1.08 | | 0.11 | | | | 0.11 | | | | — | — | — | — |
| Total impurities | Report | 1.7 | | | | 0.94 | | | | — | — | — | — |
| (% a/a) | | | | | | | | | | | | | |
| | | | Min | Max | Ave | | Min | Max | Ave | | | | |
| Dissolution (min, | Report | 10 | 95 | 99 | 97 | 10 | 97 | 100 | 98 | — | — | — | — |
| max, ave % | | 20 | 98 | 102 | 99 | 20 | 101 | 105 | 103 | | | | |
| dissolved) | | 30 | 98 | 102 | 100 | 30 | 101 | 105 | 103 | | | | |
| | | 45 | 98 | 102 | 100 | 45 | 101 | 105 | 103 | | | | |
| | | 60 | 97 | 102 | 100 | 60 | 101 | 105 | 103 | | | | |

A = Pale yellow to yellow colored, round, biconvex tablets;
HDPE = high density polyethylene;
PP = polypropylene;
CRC: Child Resistant Cap
¹ approximate RRTs,
RRT = relative retention time;
RH = relative humidity;
— scheduled testing

TABLE 12
Stability Data for Scale-Up Batch Compound 1 20 mg Tablets,
Batch #4, at 25° C./60% RH
Batch No.: #4
Storage condition: 25° C./60% RH
Container closure: 15 count in HDPE bottle with CRC/PP

| | Acceptance | Time (Months) | | | | | | | | | | | |
|---|---|---|---|---|---|---|---|---|---|---|---|---|---|
| Test | Criteria | Initial | | | | 1 | | | | 3 | 6 | 9 | 12 |
| Description | Report | A | | | | A | | | | — | — | — | — |
| Assay (% LC) | 90-110% | 99.1 | | | | 98.0 | | | | — | — | — | — |
| Water content | Report | 3.1 | | | | 3.3 | | | | — | — | — | — |
| (% w/w) | | | | | | | | | | | | | |
| Individual | | | | | | | | | | | | | |
| impurities (% a/a) | | | | | | | | | | | | | |
| RRT 0.85 | Report | 0.11 | | | | ND | | | | — | — | — | — |
| RRT 0.90 | | 0.18 | | | | ND | | | | — | — | — | — |
| RRT 1.04 | | 1.25 | | | | 0.89 | | | | — | — | — | — |
| RRT 1.08 | | 0.11 | | | | 0.11 | | | | — | — | — | — |
| Total impurities | Report | 1.7 | | | | 1.0 | | | | — | — | — | — |
| (% a/a) | | | | | | | | | | | | | |
| | | | Min | Max | Ave | | Min | Max | Ave | | | | |
| Dissolution (min, | Report | 10 | 95 | 99 | 97 | 10 | 95 | 99 | 97 | — | — | — | — |
| max, ave % | | 20 | 98 | 102 | 99 | 20 | 99 | 102 | 100 | | | | |
| dissolved) | | 30 | 98 | 102 | 100 | 30 | 99 | 102 | 100 | | | | |
| | | 45 | 98 | 102 | 100 | 45 | 99 | 102 | 100 | | | | |
| | | 60 | 97 | 102 | 100 | 60 | 99 | 102 | 100 | | | | |

A = Pale yellow to yellow colored, round, biconvex tablets;
HDPE = high density polyethylene;
PP = polypropylene;
CRC: Child Resistant Cap
¹ approximate RRTs,
RRT = relative retention time;
RH = relative humidity;
— scheduled testing

### Example 3: Manufacturing Process

### Dispensing

1. Dispense the following ingredients Compound 1, colloidal silicon dioxide, croscarmellose and magnesium stearate. Dispense microcrystalline cellulose in two portions.

### Initial and Secondary Blending

2. Screened microcrystalline cellulose (portion 1) is charged into a diffusive blender.
3. Screened Compound 1 and croscarmellose sodium is charged and blended with ingredient in step 2 for initial blending.
4. Screened microcrystalline cellulose with silicon dioxide (portion 2) is blended with ingredient in step 3 for secondary blending.

### Final Blending (Lubricated Blend)

5. Screened magnesium stearate is added to step 4 and blended to form final lubricated blend

### Tablet Compression

6. The final blend from Step 5 is compressed on rotary tablet press at target weight (50 mg for 5 mg strength and 200 mg for 20 mg strength) and target hardness and within allowed ranges.
7. Samples are taken to ensure tablets are in conformance with in-process controls for weights, hardness, friability, and visual appearance.

### Film Coating

8. Opadry 03K120035 Yellow film coating material is mixed with purified water in a tank to form film coating suspension.
9. Compressed tablets from Step 6 are loaded into a perforated pan coater and pre-warmed prior to spraying.
10. The coating suspension from Step 8 is sprayed on the rotating tablet bed of tablets in coating pan. The suspension is agitated during spraying. Spraying is continued for target weight gain of 3%.
11. After spraying is completed, coated tablets are dried at a reduced pan speed.
12. At the end of film coating, coated tablets are checked for weight. A sample is obtained for release testing.

### Bulk Packaging

13. Bulk coated tablets are packaged in plastic totes and/or fiber drums lined with two low density polyethylene (LDPE) bags.

### Bottle Packaging

14. Bulk film coated are packaged in high-density polyethylene bottles and capped with a polypropylene child-resistant (CR) closure lined induction seal liner.

### Example 4: Excipient Compatibility Study

TABLE 13
Drug excipient compatibility results

| | Composition with Compound 1 | | Vial | |
|---|---|---|---|---|
| Exp | (D:E ratio) | Week | Cap | Condition |
| 1 | Avicel PH101 (1:1) | Week-4 | Close | 40° C./ |
| 2 | Mannitol (1:1) | | | 75% RH |
| 3 | Dicalcium phosphate (1:1) | | | |
| 4 | Croscarmellose sodium (1:0.5) | | | |
| 5 | Polyplasdone XL (1:0.5) | | | |
| 6 | Klucel EXF (1:1) | | | |
| 7 | Plasdone K-90 (1:1) | | | |
| 8 | Colloidal silicon dioxide (1:0.5) | | | |
| 9 | Mg stearate (1:0.5) | | | |
| 10 | Sodium stearyl fumarate (1:0.5) | | | |
| 11 | Opadry white (1:0.5) | | | |
| 12 | Opadry II white (1:0.5) | | | |
| 13 | Opadry II brown (1:0.5) | | | |
| 14 | 10% Compound 1 | | | |
| | 85% Avicel PH101 | | | |
| | 3% Croscarmellose | | | |
| | 1% Mg stearate | | | |
| | 1% SiO2 | | | |
| 15 | 10% Compound 1 | | | |
| | 85% Mannitol | | | |
| | 3% Croscarmellose | | | |
| | 1% Sodium stearyl fumarate | | | |
| | 1% SiO2 | | | |
| 16 | 10% Compound 1 | | | |
| | 85% Dicalcium phosphate | | | |
| | 3% Croscarmellose | | | |
| | 1% Mg stearate | | | |
| | 1% SiO2 | | | |
| 1* | 10% Compound 1 | Week-2 | Close | 40° C./ |
| | 85% Mannitol | | | 75% RH |
| | 3% Polyplasdone XL | | | |
| | 1% Sodium stearyl fumarate | | | |
| | 1% SiO2 | | | |
| 17 | Avicel PH101 (1:1) | Week-4 | Open | 40° C./ |
| 18 | Mannitol (1:1) | | | 75% RH |
| 19 | Dicalcium phosphate (1:1) | | | |
| 20 | Croscarmellose sodium (1:0.5) | | | |
| 21 | Polyplasdone XL (1:0.5) | | | |
| 22 | Klucel EXF (1:1) | | | |
| 23 | Plasdone K-90 (1:1) | | | |
| 24 | Colloidal silicon dioxide (1:0.5) | | | |
| 25 | Mg stearate (1:0.5) | | | |
| 26 | Sodium stearyl fumarate (1:0.5) | | | |
| 27 | Opadry white (1:0.5) | | | |
| 28 | Opadry II white (1:0.5) | | | |
| 29 | Opadry II brown (1:0.5) | | | |
| 30 | 10% Compound 1 | | | |
| | 85% Avicel PH101 | | | |
| | 3% Croscarmellose | | | |
| | 1% Mg stearate | | | |
| | 1% SiO₂ | | | |
| 31 | 10% Compound 1 | | | |
| | 85% Mannitol | | | |
| | 3% Croscarmellose | | | |
| | 1% Sodium stearyl fumarate | | | |
| | 1% SiO₂ | | | |
| 32 | 10% Compound 1 | | | |
| | 85% Dicalcium phosphate | | | |
| | 3% Croscarmellose | | | |
| | 1% Mg stearate | | | |
| | 1% SiO2 | | | |
| 2* | 10% Compound 1 | Week-2 | Open | 40° C./ |
| | 85% Mannitol | | | 75% RH |
| | 3% Polyplasdone XL | | | |
| | 1% Sodium stearyl fumarate | | | |
| | 1% SiO2 | | | |
| 33 | Avicel PH101 (1:1) | Week-4 | Close | 50° C. |
| 34 | Mannitol (1:1) | | | |
| 35 | Dicalcium phosphate (1:1) | | | |
| 36 | Croscarmellose sodium (1:0.5) | | | |
| 37 | Polyplasdone XL (1:0.5) | | | |
| 38 | Klucel EXF (1:1) | | | |
| 39 | Plasdone K-90 (1:1) | | | |
| 40 | Colloidal silicon dioxide (1:0.5) | | | |
| 41 | Mg stearate (1:0.5) | | | |
| 42 | Sodium stearyl fumarate (1:0.5) | | | |
| 43 | Opadry white (1:0.5) | | | |
| 44 | Opadry II white (1:0.5) | | | |
| 45 | Opadry II brown (1:0.5) | | | |
| 46 | 10% Compound 1 | | | |
| | 85% Avicel PH101 | | | |
| | 3% Croscarmellose | | | |
| | 1% Mg stearate | | | |
| | 1% SiO₂ | | | |
| 47 | 10% Compound 1 | | | |
| | 85% Mannitol | | | |
| | 3% Croscarmellose | | | |
| | 1% Sodium stearyl fumarate | | | |
| | 1% SiO₂ | | | |
| 48 | 10% Compound 1 | | | |
| | 85% Dicalcium phosphate | | | |
| | 3% Croscarmellose | | | |
| | 1% Mg stearate | | | |
| | 1% SiO₂ | | | |
| 3* | 10% Compound 1 | Week-2 | Close | 50° C. |
| | 85% Mannitol | | | |
| | 3% Polyplasdone XL | | | |
| | 1% Sodium stearyl fumarate | | | |
| | 1% SiO₂ | | | |

TABLE 14
HPLC results of drug excipient compatibility results
HPLC Results

| | | % Area | | | |
|---|---|---|---|---|---|
| | | Imp-1- | Imp-2- | | Imp-3- |
| | | RRT- | RRT- | | RRT- |
| Exp. | Condition | 0.780 | 0.797 | API | 1.03 |
| Compound 1-Inj-1- | N/A | 0.21 | 0.26 | 97.77 | 1.76 |
| untreated conc. | | | | | |
| 0.4 mg/mL | | | | | |
| 1 | 40° C./ | 0.17 | 0.18 | 98.20 | 1.45 |
| 2 | 75% RH | 0.21 | 0.20 | 98.02 | 1.57 |
| 3 | Closed | 0.15 | 0.17 | 98.36 | 1.32 |
| 4 | | 0.23 | 0.19 | 97.79 | 1.79 |
| 5 | | 0.23 | 0.20 | 97.89 | 1.68 |
| 6 | | 0.24 | 0.21 | 97.80 | 1.75 |
| 7 | | 0.16 | 0.19 | 97.91 | 1.73 |
| 8 | | 0.19 | 0.17 | 98.04 | 1.60 |
| 9 | | 0.00 | 0.09 | 98.14 | 0.77 |
| 10 | | 0.11 | 0.14 | 98.01 | 1.74 |
| 11 | | 0.00 | 0.10 | 98.40 | 1.49 |
| 12 | | 0.10 | 0.13 | 98.03 | 1.74 |
| 13 | | 0.18 | 0.15 | 97.96 | 1.71 |
| 14 | | 0.00 | 0.12 | 98.77 | 1.11 |
| 15 | | 0.00 | 0.10 | 98.19 | 1.70 |
| 16 | | 0.00 | 0.10 | 98.26 | 1.64 |
| 1* | | 0.10 | 0.14 | 98.05 | 1.72 |
| 17 | 40° C./ | 0.00 | 0.12 | 98.38 | 1.50 |
| 18 | 75% RH | 0.10 | 0.14 | 98.19 | 1.57 |
| 19 | Open | 0.14 | 0.15 | 98.10 | 1.61 |
| 20 | | 0.09 | 0.11 | 98.00 | 1.80 |
| 21 | | 0.00 | 0.12 | 98.22 | 1.66 |
| 22 | | 0.00 | 0.14 | 98.20 | 1.65 |
| 23 | | 0.10 | 0.13 | 98.07 | 1.71 |
| 24 | | 0.09 | 0.14 | 98.08 | 1.69 |
| 25 | | 0.00 | 0.09 | 98.09 | 1.82 |
| 26 | | 0.00 | 0.11 | 98.26 | 1.64 |
| 27 | | 0.09 | 0.12 | 98.13 | 1.66 |
| 28 | | 0.12 | 0.12 | 98.21 | 1.54 |
| 29 | | 0.00 | 0.10 | 98.24 | 1.66 |
| 30 | | 0.00 | 0.10 | 98.20 | 1.69 |
| 31 | | 0.00 | 0.08 | 98.42 | 1.50 |
| 32 | | 0.10 | 0.13 | 98.12 | 1.65 |
| 2* | | 0.00 | 0.13 | 98.25 | 1.62 |
| 33 | 50° C. | 0.11 | 0.17 | 98.12 | 1.61 |
| 34 | | 0.00 | 0.14 | 98.16 | 1.70 |
| 35 | | 0.00 | 0.14 | 98.05 | 1.81 |
| 36 | | 0.20 | 0.18 | 97.89 | 1.72 |
| 37 | | 0.21 | 0.18 | 97.85 | 1.76 |
| 38 | | 0.00 | 0.13 | 98.20 | 1.67 |
| 39 | | 0.23 | 0.19 | 97.98 | 1.60 |
| 40 | | 0.15 | 0.13 | 98.06 | 1.66 |
| 41 | | 0.16 | 0.19 | 97.96 | 1.69 |
| 42 | | 0.00 | 0.09 | 98.38 | 1.53 |
| 43 | | 0.00 | 0.10 | 98.24 | 1.66 |
| 44 | | 0.00 | 0.10 | 98.31 | 1.59 |
| 45 | | 0.00 | 0.11 | 98.90 | 0.99 |
| 46 | | 0.00 | 0.11 | 98.33 | 1.55 |
| 47 | | 0.00 | 0.13 | 98.61 | 1.26 |
| 48 | | 0.17 | 0.19 | 97.89 | 1.75 |
| 3* | | 0.11 | 0.17 | 98.12 | 1.61 |

Partition Coefficient Determination by Shake Flask Method

The examples and embodiments described herein are for illustrative purposes only and in some embodiments, various modifications or changes are to be included within the purview of disclosure and scope of the appended claims.

What is claimed is:

1. A pharmaceutical composition comprising:
(a) about 5% to about 20% (w/w) 5-((5-(4-(((1R,3S)-3-aminocyclopentyl)oxy)-2-methoxy-6-methylpyridin-3-yl)-1H-pyrazol-3-yl)amino)pyrazine-2-carbonitrile: 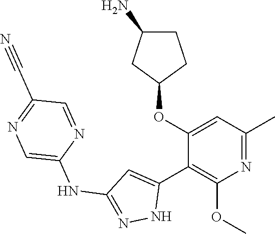 [SMILES: COc1nc(C)cc(O[C@@H]2CC[C@H](N)C2)c1-c1cc(Nc2cnc(C#N)cn2)n[nH]1]
(Compound 1) or a pharmaceutically acceptable salt thereof;
(b) about 70% to about 90% (w/w) of a diluent;
(c) about 1% to about 10% (w/w) of a disintegrant;
(d) about 0.1% to about 2% of a glidant; and
(e) about 0.1% to about 2% of a lubricant.

2. The pharmaceutical composition of claim 1, wherein the amount of the diluent in the pharmaceutical composition is between about 80% and about 90% (w/w).

3. The pharmaceutical composition of claim 1 or 2, wherein the amount of the diluent in the pharmaceutical composition is about 85% (w/w).

4. The pharmaceutical composition of any one of claims 1-3, wherein the diluent is microcrystalline cellulose.

5. The pharmaceutical composition of any one of claims 1-4, wherein the amount of the disintegrant in the pharmaceutical composition is between about 2% to about 4% (w/w).

6. The pharmaceutical composition of any one of claims 1-5, wherein the amount of the disintegrant in the pharmaceutical composition is about 3% (w/w).

7. The pharmaceutical composition of any one of claims 1-6, wherein the disintegrant is croscarmellose sodium.

8. The pharmaceutical composition of any one of claims 1-7, wherein the amount of the glidant in the pharmaceutical composition is between about 0.5% to about 1.5% (w/w).

9. The pharmaceutical composition of any one of claims 1-8, wherein the amount of the glidant in the pharmaceutical composition is about 1% (w/w).

10. The pharmaceutical composition of any one of claims 1-9, wherein the glidant is colloidal silicon dioxide.

11. The pharmaceutical composition of any one of claims 1-10, wherein the amount of the lubricant in the pharmaceutical composition is between about 0.5% to about 1.5% (w/w).

12. The pharmaceutical composition of any one of claims 1-11, wherein the amount of the lubricant in the pharmaceutical composition is about 1% (w/w).

13. The pharmaceutical composition of any one of claims 1-12, wherein the lubricant is magnesium stearate.

14. The pharmaceutical composition of any one of claims 1-13, further comprising a coating.

15. The pharmaceutical composition of claim 14, wherein the coating comprises a film former, a plasticizer, an opacifying agent, a colorant, or any combination thereof.

16. The pharmaceutical composition of any one of claims 1-15, wherein the pharmaceutical composition is stable at 25° C./60% RH.

17. The pharmaceutical composition of any one of claims 1-15, wherein the pharmaceutical composition is stable at 40° C./75% RH.

18. The pharmaceutical composition of any one of claims 1-15, wherein the pharmaceutical composition is stable at 2-8° C.

19. The pharmaceutical composition of any one of claims 1-18, wherein the pharmaceutical composition is comprised in a tablet.

20. The pharmaceutical composition of any one of claims 1-19, wherein the pharmaceutical composition is comprised in a 5 mg tablet or a 20 mg tablet.

21. A method of treating cancer in a subject in need thereof comprising administering to the subject a pharmaceutical composition of any one of claims 1-20.

22. The method of claim 21, wherein the cancer comprises a solid tumor.

23. The method of claim 22, wherein the cancer comprises a locally advanced or metastatic non-resectable solid tumor.

24. The method of any one of claims 21-23, wherein the cancer comprises a tumor or tumor cells harboring an oncogene amplification.

25. The method of claim 24, wherein the oncogene amplification comprises an amplification of ABL, AKT1, AKT2, ALK, androgen receptor, BRAF, CCND1, CCND2, CCND3, CCNE1, CDK12, CDK4, CDK6, EGFR, ERBB2, EZH2, FGFR1, FGFR2, FGFR3, FLT3, IDH1/2, JAK2, JAK3, KIT, KRAS, MDM2, MDM4, MET, MYC, MYCL, MYCN, NRAS, PDGFRA, TERT, VEGFRA, or any combination thereof.

26. The method of claim 24 or claim 25, wherein the oncogene amplification resides on ecDNA.

27. The method of claim 24 or claim 25, wherein the oncogene amplification resides on one or more chromosomal loci.

28. The method of claim 24 or claim 25, wherein the oncogene amplification is an ecDNA-derived amplification.

29. The method of any one of claims 21-28, wherein the pharmaceutical composition is administered every other day.

30. The method of any one of claims 21-29, wherein the cancer is an ovarian cancer.

31. The method of claim 30, wherein the ovarian cancer is a platinum resistant high-grade serous ovarian cancer, a primary peritoneal cancer, or a fallopian tube cancer.

32. The method of any one of claims 21-29, wherein the cancer is a uterine cancer.

33. The method of claim 30, wherein the uterine cancer is a high-grade endometrial carcinoma, a uterine serous carcinoma or a uterine carcinosarcoma.

34. The method of any one of claims 21-29, wherein the cancer is colorectal cancer, esophageal cancer, gastric cancer, gastroesophageal junction (GEJ) cancer, head and neck squamous cell carcinoma, liposarcoma, non-small cell lung cancer, or subtype squamous cell carcinoma.

35. The method of any one of claims 21-29, wherein the cancer is glioblastoma or a neuroblastoma.

36. The method of any one of claims 21-29, wherein the cancer is breast cancer, cholangiocarcinoma, esophageal cancer, neck squamous cell carcinoma, non-small cell lung cancer, stomach cancer, or subtype squamous cell carcinoma.

37. The method of any one of claims 21-29, wherein the cancer is esophageal cancer, non-small cell lung cancer, a sarcoma, or stomach cancer.

38. The method of any one of claim 21-37, wherein the treatment further comprises administering an additional therapeutic agent.

39. The method of claim 38, wherein the oncogene amplification comprises CDK4, CDK6, EGFR, FGFR1, FGFR2, or FGFR3.

40. The method of claim 39, wherein the treatment further comprises administering a CDK4/6 inhibitor, an EGFR inhibitor, or a FGFR inhibitor.

41. The method of claim 40, wherein the EGFR inhibitor is erlotinib.

42. The method of claim 40, wherein the FGFR inhibitor is pemigatinib.

43. The method of claim 40, wherein the FGFR inhibitor is futibatinib.

44. The method of claim 40, wherein the CDK4/6 inhibitor is abemaciclib.

45. The method of any one of claims 38-44, wherein the pharmaceutical composition of Compound 1 and the additional therapeutic agent are each formulated for oral administration.
